(19) United States
(12) Patent Application Publication
(10) Pub. No.: US 2025/0255841 A1
(43) Pub. Date: Aug. 14, 2025

(54) METHODS FOR TREATING CYTOKINE STORM ASSOCIATED WITH ACINETOBACTER BAUMANNII INFECTIONS

(71) Applicant: City University of Hong Kong, Hong Kong (CN)

(72) Inventors: Han Wang, Hong Kong (CN); Qi XU, Hong Kong (CN); Guan YANG, Hong Kong (CN); Sheng CHEN, Hong Kong (CN)

(21) Appl. No.: 18/602,132

(22) Filed: Mar. 12, 2024

Related U.S. Application Data
(60) Provisional application No. 63/551,622, filed on Feb. 9, 2024.

(51) Int. Cl. A61K 31/192; A61K 45/06; A61P 37/06

(52) U.S. Cl. CPC A61K 31/192; A61K 45/06; A61P 37/06

(57) ABSTRACT

Methods for treating a cytokine storm in a subject in need thereof, the method comprising: administering a therapeutically effective amount of a therapeutic selected from the group consisting of (+)-(S)-2-(6-methoxynaphthalen-2-yl) propanoic acid, acetylsalicylic acid, dexamethasone, azathioprine and pharmaceutically acceptable salts thereof to the subject, wherein the cytokine storm is associated with an Acinetobacter baumannii infection in the subject.

### CROSS-REFERENCE TO RELATED APPLICATIONS

The present application claims priority from U.S. Provisional Patent Application No. 63/551,622, filed on Feb. 9, 2024, which is hereby incorporated by reference in its entirety.

### REFERENCE TO SEQUENCE LISTING

The Sequence Listing identified as Sequence_Listing_P25718US00.xml; Size: 30.5 kilobytes; and Date of Creation: Jan. 24, 2024, filed herewith, is hereby incorporated by reference in its entirety.

### TECHNICAL FIELD

The present disclosure provides methods of treating cytokine storms associated with Acinetobacter baumannii infections in a subject in need thereof.

### BACKGROUND

Acinetobacter baumannii (Ab) is an opportunistic bacterial pathogen that primarily infects immunocompromised individuals. Hospital infections caused by Ab have become a global health concern in recent years due to the emergence of multidrug-resistant (MDR) strains 1,2. This pathogen can cause infections at various body sites, such as skin, soft tissue, and the urinary tract. More serious infections including ventilator-related pneumonia and sepsis are associated with an unacceptably high mortality rate 3,4. The difficulty in treating clinical Ab infections has further increased due to the continuous evolution of drug-resistance phenotypes in this organism. A lack of active antimicrobials has prompted the development of novel therapies to control these infections. Furthermore, there is currently only superficial understanding of the nature of interaction between Ab and the human host. Therefore, comprehensive investigation of the Ab-induced immune response in the human body can help identify potential targets for development of novel approaches for treatment of Ab infections.

Current data show that Ab infection-induced sepsis is attributed to dysregulation of the innate immune system, the function of which is mediated by pattern recognition receptors (PRRs). It is therefore necessary to maintain a delicate balance between inflammatory and anti-inflammatory responses^{5,6}. Several studies showed that pro-inflammatory cytokines are beneficial to Ab clearance and infection control^{7,8}. However, excessive inflammatory response can lead to onset of systemic inflammatory response syndrome, organ damage, and even death⁹. Toll-like receptors (TLRs) and other PRRs are pivotal to initiating the immune response but are also responsible for regulating the degree of inflammatory response by recognizing specific pathogen-associated molecular patterns (PAMPs) of Ab^{10,11}. Therefore, it is imperative to gain a thorough understanding of Ab-host interactions and the mechanism underlying production of cytokines and regulation of inflammatory reactions during infection by Ab. Also, there is thus a need for improved methods for treating an Ab associated cytokine storm in a subject in need thereof.

### SUMMARY

As disclosed herein, it has been discovered that alveolar and interstitial macrophages are the key components of the host immune system that regulate the changes in Ab-induced inflammatory microenvironment. These macrophages can undergo polarization toward the M1 phenotype and cause onset of cytokine storm, often resulting in death of the host. Furthermore, we found that the M1 polarization event triggered by Ab was mainly mediated by the TLR2/MyD88/NF-κB signaling pathway, and that this even would result in cytokine over-production. Importantly, it is shown that the drugs naproxen (NPXS), acetylsalicylic acid (ASA), dexamethasone (DXMS), and azathioprine (AzA) could effectively suppress excessive inflammatory response in infected mice by inhibiting the TLR2/MyD88/NF-κB signaling pathway, thereby protecting the infected mice from death. These findings suggest that the use of immunosuppressive drugs (NPXS) represents a promising therapeutic strategy for management of acute Ab infection.

In a first aspect, provided herein is a method of treating a cytokine storm in a subject in need thereof, the method comprising: administering a therapeutically effective amount of a therapeutic agent selected from the group consisting of (+)-(S)-2-(6-methoxynaphthalen-2-yl) propanoic acid (naproxen), acetylsalicylic acid (ASA), dexamethasone (DXMS), azathioprine (AzA), and pharmaceutically acceptable salts thereof to the subject, wherein the cytokine storm is associated with an Acinetobacter baumannii infection in the subject.

In certain embodiments, the therapeutic is naproxen or a pharmaceutically acceptable salt thereof.

In certain embodiments, the Acinetobacter baumannii infection activates TLR2/MyD88/NF-κB signaling pathway in the subject.

In certain embodiments, the subject overexpresses one or more pro-inflammatory factors selected from the group consisting of IL-18, IL-6, IL-10, IL-12, IL17a, IL23, IL-27, IFN-γ, and TNF-α.

In certain embodiments, the Acinetobacter baumannii infection activates a TLR2/MyD88/NF-κB signaling pathway in the subject; the therapeutic is naproxen or a pharmaceutically acceptable salt thereof; and administration of naproxen results in a reduction in the relative expression of TLR2/MyD88/NF-κB signaling pathway-related genes.

In certain embodiments, the TLR2/MyD88/NF-κB signaling pathway-related genes are selected from the group consisting of toll-like receptor 2 (TLR2), myeloid differentiation primary response 88 (Myd88), nuclear factor kappa B subunit 1 (Nfkb1), nuclear factor kappa B subunit 2 (Nfkb2), Interleukin 1 beta (Il1b), interleukin 6 (Il6), and tumor necrosis factor (Tnf).

In certain embodiments, the Acinetobacter baumannii infection is the result of antibiotic resistant Acinetobacter baumannii.

In certain embodiments, the antibiotic resistant Acinetobacter baumannii is resistant to one or more antibacterials selected from the group consisting of aminoglycosides, fluoroquinolones, and carbapenems.

In certain embodiments, the therapeutic is naproxen or a pharmaceutically acceptable salt thereof and the Acinetobacter baumannii infection is the result of antibiotic resistant Acinetobacter baumannii.

In certain embodiments, the Acinetobacter baumannii infection results from an Acinetobacter baumannii strain selected from the group consisting of ATCC 17978, ATCC 19606, AB5075, ATCC 9955, ATCC 17904, R 477, and R 0211019.

In certain embodiments, the therapeutic is naproxen or a pharmaceutically acceptable salt thereof, and the Acinetobacter baumannii infection results from an Acinetobacter baumannii strain selected from the group consisting of ATCC 17978, ATCC 19606, AB5075, ATCC 9955, ATCC 17904, R 477, and R 0211019.

In certain embodiments, the Acinetobacter baumannii infection is present in one or more of a wound, a surgical site, a catheter site, blood, urinary tract, skin, lungs, or respiratory tract.

In certain embodiments, the method further comprises diagnosing the subject with an Acinetobacter baumannii infection prior to administering the therapeutically effective amount of naproxen.

In certain embodiments, the method further comprises co-administering a therapeutically effective amount of an antibacterial agent to the subject.

In certain embodiments, the antibacterial agent is selected from the group consisting of meropenem, polymyxin E, polymyxin B, sulbactam, piperacillin/tazobactam, minocycline, tigecycline and aminoglycosides.

### BRIEF DESCRIPTION OF THE DRAWINGS

The above and other objects and features of the present disclosure will become apparent from the following description of the disclosure, when taken in conjunction with the accompanying drawings.

FIG. 1. Assessment of virulence level of Ab in a mouse sepsis model. (A) Kaplan-Meier survival curves of mice (n=5 per group) infected by Ab strains. (B) Relative body weight loss of Ab-infected mice at 12 hpi. (C) Percentage of spleen weight and representative image of spleens in mice after Ab inoculation. (D) Bacteria loads in the lungs, spleen, kidney, livers and blood samples of mice at 12 hpi. (E) Survival of intracellular bacteria obtained from lungs and spleen samples of the infected mice at 12 hpi. *p<0.05, **p<0.01; ***p<0.001; ****p<0.0001; ns, significant.

FIG. 2. Ab-triggered cytokine storm in the host. (A) The percentages of F4/80+ macrophages and Ly6G+ neutrophils gated on total CD45+ cells in lung cells analyzed by flow cytometry. (B) Quantification of total F4/80+ and Ly6G+ cells and expression of the surface CD86 and CD206 on F4/80+ macrophages presented by mean fluorescence intensity (MFI) values in the lung cells. (C) The percentages of CD3+ T cells, CD19+B cells, NK1.1+ NK cells gated on total CD45+ cells. (D) Quantification of total CD3+, CD19+, NK1.1+ cells and lymphocytes. (E) KEGG enrichment analysis depicts the most affected pathways in Ab infection. (F and G) Heatmap which shows the differential expression patterns underlying M1 polarization (F) and production of chemokines (G) in healthy and Ab-infected mice. (H) The transcription levels of proinflammation (up) and chemokines (down) in different mouse groups. (I) IL-1b, IL-6, TNF-α expression levels in the Ab-infected and non-infected mice serum. ** p<0.01; ***p<0.001; ****p<0.0001.

FIG. 3. The molecular cell atlas and cell-to-cell signaling networks of lung cells depicted on the basis of single-cell RNA sequencing data. (A) The UMAP projection of cells from the lungs of healthy mice and ATCC 17978-infected mice shows the main clusters and respective cell-type assignments. Each dot corresponds to an individual cell. Different colors represent different cell clusters. (B) Origins of cells with the same embedding as in (A). Distinct colors depict infected and non-infected lung cells. (C) Total RNA expression of Plet1 and Csf1r in the same embedding as (A). (D) Frequency of detection of different types of cells shown in (A). (E) Differences in overall information flow in interaction networks between healthy and Ab-infected mice. The red-labeled signaling pathways were more enriched in infected individuals, the black-labeled signaling pathway was equally enriched in the two groups; the blue-labeled signaling pathway was more enriched in healthy individuals. (F and G) Circular plots showing the inferred intercellular communication network of the TNF signaling pathway (F) and TGFb signaling pathway (G) in healthy mice (left) and ATCC 17978-infected mice (right). The circle sizes are proportional to the number of cells in each cell group, and the edge width represents the communication probability. The edge colors correspond to the signaling source.

FIG. 4. Ab mediated onset of cytokine storm by inducing M1 polarization. (A) UMAP of AMs and the corresponding group assignments. (B and C) Pseudo-time trajectory of total AM cells colored by pseudotime (B) and different samples (C). (D) Cxcl2 expression of healthy and infected mice in the same embedding as (A). (E) Percentage of Cxcl2+ and CD36+ cells in AMs. (F) UMAP of IMs with cell-type assignments. (G and H) Pseudo-time trajectory of total IM cells colored by pseudotime (G) and different samples (H). (I) Il1b expression of healthy and infected mice in the same embedding as (F). (J) Percentage of Tnf+ and Gadd45g+ cells shown in IMs. (K and L) KEGG enrichment analysis showing the most affected pathways in AMs (K) and IMs (L) upon Ab infection.

FIG. 5. Blocking of the TLR2/Myd88/NF-κB pathway attenuated M1 polarization and cytokine storm caused by Ab infection. (A) Heatmap that depicts the differential expression profile of toll-like receptor (TLR) and the TLR2/Myd88/NF-κB pathway in AMs and IMs of healthy and Ab-infected mice. (B) The relative transcription levels of genes involved in the TLR2/Myd88/NF-κB pathway in different mice groups. (C) The TLR2 MFI on the surface of F4/80+ macrophages in the lungs of infected mice. (D) Pseudo-time trajectory of total cells colored by Tlr2, Myd88, Nfkb1, Nfkb2, Il1b, Il6, Tnf expression levels in IMs and AMs. (E and F) The CD86 (E) and CD206 (F) MFI on the surface of RAW264.7 cells pretreated with culture media, C29, TJ-M2010-5 or JSH-23, and then infected with ATCC 17978. (G and H) TNF-α (G) and IL-6 (H) levels in supernatant of Ab-infected RAW264.7 cells pretreated with medium, C29, TJ-M2010-5 or JSH-23. (I) Kaplan-Meier survival curves of WT and TLR2−/− mice infected by ATCC17978. (J) The percentages of F4/80+ macrophages gated on total CD45+ cells determined by flow cytometry. (K) Quantification of total F4/80+ cells analyzed in (J). (L) The surface CD86 expression on F4/80+ macrophages in the lungs. (M and N) The relative transcription levels of proinflammation (M) and chemokines (N) in different mice groups. (O) IL-1b, IL-6, TNF-α expression levels in the healthy, infected WT and infected TLR2−/− mice serum. (P) mRNA expression of Tlr2 in RAW264.7 incubated with the strains ATCC 17978 and ΔOmpA (MOI 5) for 3 h. (Q, R and S) The adhesion rate (Q), invasion rate (R) and intracellular survival efficiency(S) of strain ATCC 17978 and ΔOmpA in macrophages. *p<0.05, **p<0.01; ***p<0.001; ****p<0.0001; ns, not significant.

FIG. 6. NPXS suppressed cytokine storm caused by Ab infection. (A) Kaplan-Meier survival curves of Ab-infected mice treated with immunosuppressive drugs. (B) Relative body weight loss of healthy, Ab-infected and NPXS treated Ab-infected mice at 12 hpi. (C) Percentage of spleen weight and representative image of spleens in healthy, ATCC 17978-infected, and ATCC 17978-infected+NPXS treated mice. (D) The percentages of F4/80⁺ macrophages gated on total CD45+ cells determined by flow cytometry. (E) Quantification of total F4/80+ cells analyzed in (D) and the surface CD86 expression on F4/80⁺ macrophages in the lungs. (F) Heatmap showing the differential expression profile of proinflammation marker genes in control, ATCC 17978-infected, and ATCC 17978-infected+NPXS treated mice. (G) mRNA expression of M1 markers and proinflammatory cytokines in lungs of mice subjected to different treatments. (H) Quantitation of TNF-α, IL-1b, and IL-6 production in serum analyzed by ELISA. (I) Heatmap of the TLR2/Myd88/NF-κB signaling pathway in mice of three groups. (J) Relative transcription levels of the TLR2/Myd88/NF-κB signaling pathway-related genes. *p<0.05, **p<0.01; ***p<0.001; ****p<0.0001; ns, not significant.

FIG. 7. Differential gene expression analysis of lung cells of healthy and infected mice by RNA-seq. (A) The volcano plot of differentially expressed genes. Heatmap of interleukins (B), TNF family clusters (C), and neutrophil activation markers (D) of DEGs.

FIG. 8. Differential gene expression analysis of Ly6G+ cells of healthy and Ab-infected mice. Heatmap depicting relative gene expression levels of TLR (A), genes of the TLR2/Myd88/NF-κB pathway (B) and the neutrophil activation markers (C). (D) KEGG enrichment analysis showing the most affected pathways after Ab infection.

FIG. 9. Deletion of the ompA gene in the ATCC17978 strain. (A) Colony PCR results showing that only a scar sequence remains at the locus in mutant stain. The lane of CK represents the PCR band from the wild-type ATCC 17978 strain which serves as the control, and lanes 1, 2, 4, 5, 6, and 7 are mutants in which the ompA gene has been successfully deleted. (B) The sequencing result of the mutant site in ATCC 17978 and ΔompA.

FIG. 10. Depicts a table showing strains and plasmids used in this study.

FIG. 11. Depicts a table showing the primers and ssDNA used in this study.

### DETAILED DESCRIPTION

### Definitions

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

The term “therapeutically effective amount” as used herein, means that amount of the compound or therapeutic agent that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The terms “co-administration” and “co-administering” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent at the same time.

As used herein, the term “subject” refers to any animal (e.g., a mammal), including, but not limited to, humans, non-human primates, canines, felines, and rodents.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, non-toxic ammonium, quaternary ammonium, and amine cations formed using counterions, such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In certain embodiments, the pharmaceutically acceptable base addition salt is chosen from ammonium, potassium, sodium, calcium, and magnesium salts.

The present disclosure provides method of treating a cytokine storm in a subject in need thereof, the method comprising: administering a therapeutically effective amount of a the therapeutic selected from the group consisting of naproxen, ASA, DXMS, AzA, and pharmaceutically acceptable salts thereof to the subject, wherein the cytokine storm is associated with an Acinetobacter baumannii infection in the subject. In certain embodiments, the therapeutic is naproxen.

In certain embodiments, the Acinetobacter baumannii infection activates the subject's TLR2/MyD88/NF-κB signaling pathway, which can increase TLR2, MyD88 and NF-κB mRNA and/or protein in the subject.

In certain embodiments, the Acinetobacter baumannii infection gives rise to overexpression of one or more pro-inflammatory factors selected from the group consisting of IL-18, IL-6, IL-10, IL-12, IL17a, IL23, IL-27, IFN-γ, and TNF-α.

Administration of a therapeutically effective amount of naproxen to the subject can result in a reduction in the relative expression of TLR2/MyD88/NF-κB signaling pathway-related genes, such as toll-like receptor 2 (TLR2), myeloid differentiation primary response 88 (Myd88), nuclear factor kappa B subunit 1 (Nfkb1), nuclear factor kappa B subunit 2 (Nfkb2), Interleukin 1 beta (Il1b), interleukin 6 (Il6), and tumor necrosis factor (Tnf), which can result in a reduction of TLR2, Myd88, Nfkb1, Nfkb2, Il1b, 116, and Tnf mRNA and proteins.

The Acinetobacter baumannii infection can be an infection of the skin (including but not limited to skin ulcers, bed sores, diabetic foot ulcers, etc), infections in and around wounds, post-operative infections, infections associated with catheters, surgical drains, other medical/diagnostic implements and medical procedures, infections of the blood, infections of the respiratory tract, infections of the cerebrospinal fluid, infections of peritoneal fluid, and infections of the urinary tract.

In certain embodiments, the Acinetobacter baumannii infection results from an Acinetobacter baumannii strain selected from the group consisting of AB001, AB002, AB0057, AB967, AB2828, AB3340, AB3560, AB3638, AB3785, AB3806, AB3917, AB3927, AB402S, AB4026, AB4027, AB4052, AB4269, AB4448, AB4456, AB4490, AB4498, AB4795, AB4857, AB4878, AB4932, AB4957, AB4991, ABS001, ABS075, AB5197, AB5256, AB5674, ABS711, ATCC 9955, ATCC 17904, ATCC 17978, ATCC 19606, R 477, R 0211019, RUH134, RUH875, RUH5875, SDF, AYE, or ACICU. In certain embodiments, the Acinetobacter baumannii infection results from an Acinetobacter baumannii strain selected from the group consisting of ATCC 17978, ATCC 19606, and AB5075.

In certain embodiments, the Acinetobacter baumannii infection results from antibiotic resistant Acinetobacter baumannii. In certain embodiments, the antibiotic resistant Acinetobacter baumannii is resistant to one or more antibiotics selected from the group consisting of colistin, erythromycin, ampicillin, vancomycin, linezolid, methicillin, oxacillin, ceotaxime, rifampicin, amikacin, gentamicin, kanamycin, tobramycin, neomycin, ertapenem, doripenem, imipenem, meropenem, ceftazidime, cefepime, ceftaroline, ceftobiprole, aztreonam, piperacillin, polymyxin B, ciprofloxacin, levofloxacin, moxifloxacin, gatifloxacin, piperacillin, minocycline, tigecycline, cotrimoxa, and derivatives thereof. In certain embodiments, the Acinetobacter baumannii is multi-drug resistant Acinetobacter baumannii, which is resistant to aminoglycosides, fluoroquinolones, and carbapenems.

In certain embodiments, the method described herein further comprises testing the subject for an Acinetobacter baumannii infection or diagnosing a subject with Acinetobacter baumannii infection. In certain embodiments, the step of diagnosing the subject with an Acinetobacter baumannii infection comprises providing a sample from the subject, conducting a bacteria culture on the sample, identifying the bacteria in the bacteria culture, and diagnosing the subject with Acinetobacter baumannii infection based on the identified bacteria. The sample can comprise or can be derived from a blood, stool, urine, mucus, spinal fluid, or other bodily fluid sample obtained from the subject.

The method described herein can be used in combination with the standard of care for treating Acinetobacter baumannii infections. Accordingly, in certain embodiments, the method described herein further comprises co-administering a therapeutically effective amount of an antibacterial agent to the subject.

Any antibacterial agent that is useful in the treatment of an Acinetobacter baumannii infection may be used. The selection of the appropriate antibacterial agent is well within the skill of a person of ordinary skill in the art. Exemplary antibacterial agents include, but are not limited to carbapenems, such as meropenem, polymyxin E (colistin), polymyxin B, sulbactam, piperacillin/tazobactam, minocycline, tigecycline and aminoglycosides.

### Results

### Establishing an Ab Infection Sepsis Model in Mice

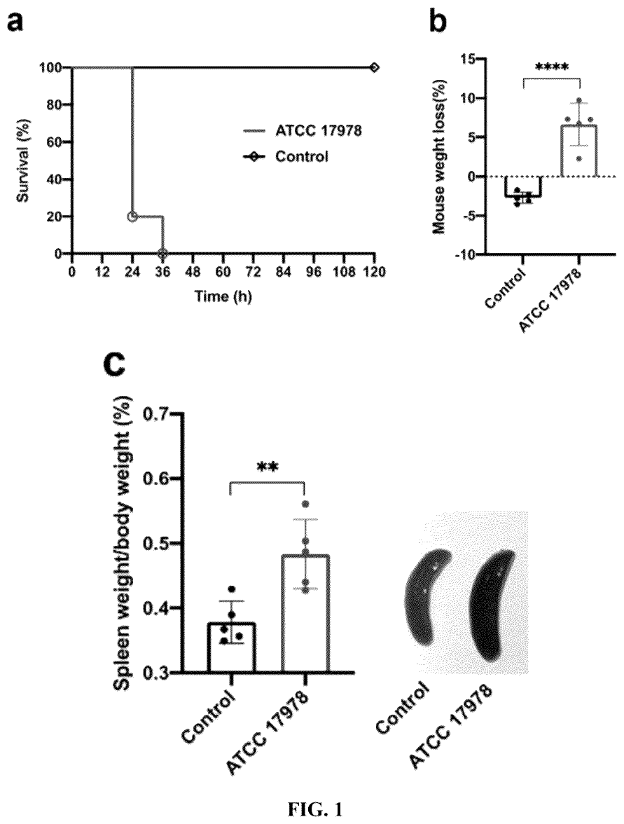

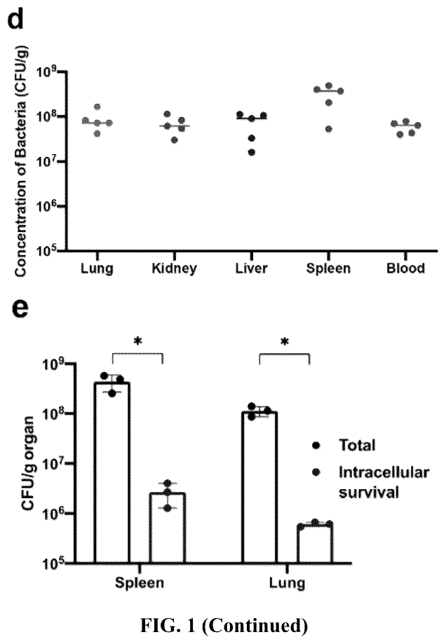

To investigate the lethality and pathogenesis of Ab, a mouse sepsis infection model was established. Briefly, following i.v. infection with 7.5×10⁷ CFU of ATCC strain 17978, all mice exhibited mortality within 36 hours (FIG. 1A). At 12-hour post-infection (hpi), poor health status, decreased body weight, and splenomegaly was observed in each mouse (FIGS. 1, B and C). At 12 hpi, the lungs, spleen, liver, kidney and blood samples were collected from the infected mice; the bacterial load in these samples was counted and found to be as high as 10⁷-10⁹ CFU/g (FIG. 1D). To assess the intracellular survival of Ab, cells obtained from the lungs and spleen samples were incubated with 300 μg/mL gentamicin to eliminate extracellular bacteria. A significant number of ATCC 17978 was found to remain viable at 12 hpi in phagocytes; the total bacterial load was found to be higher than the intracellular population, indicating that this strain could survive in both intra- and extracellular environments (FIG. 1E), and that strain ATCC 17978 in the mouse sepsis model could not be effectively cleared by the immune system.

### Ab Infection-Induced Fluctuations in Immune Response and Onset of Cytokine Storm in Mice

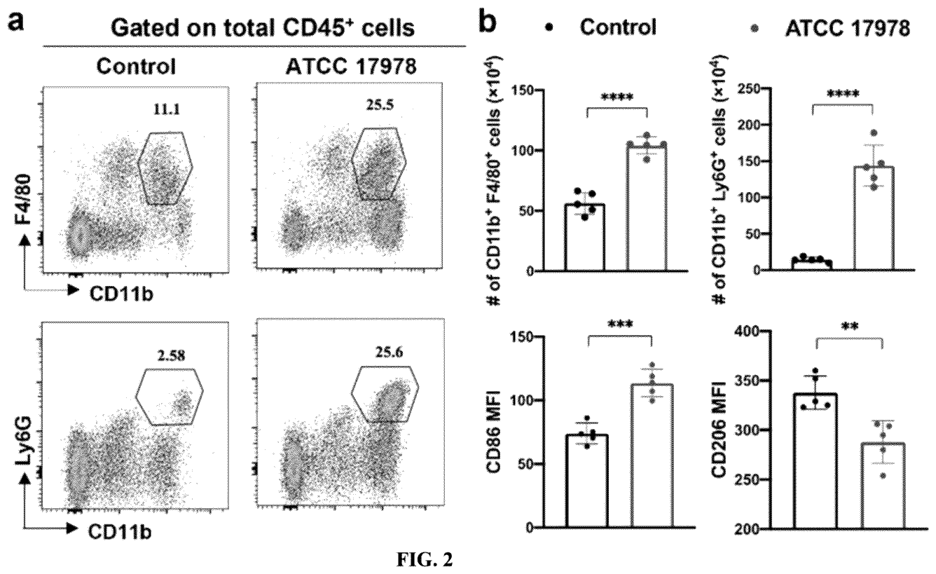

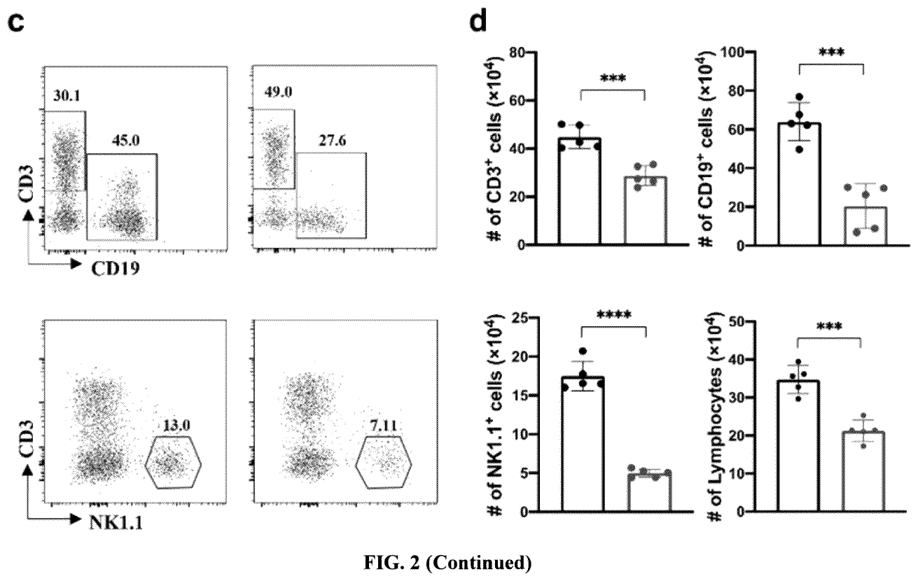

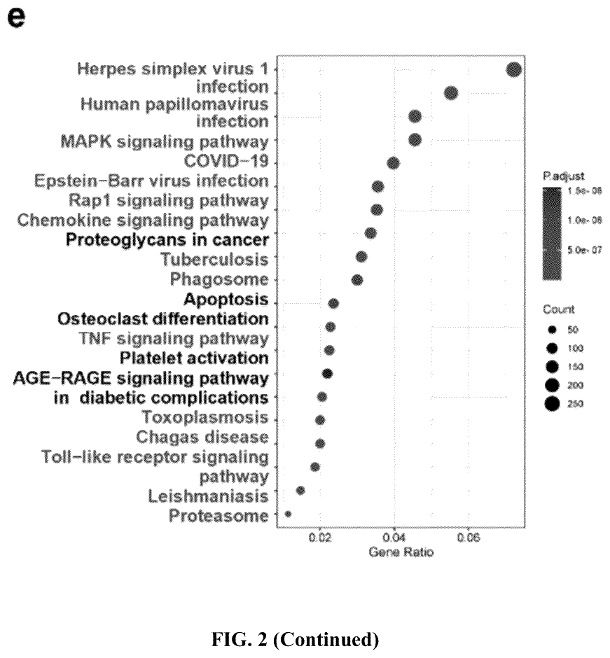

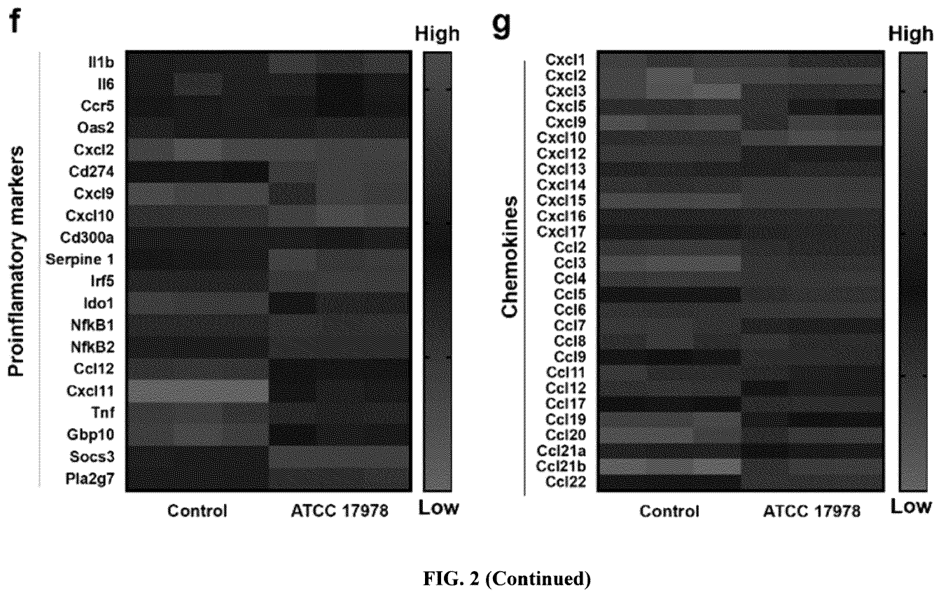

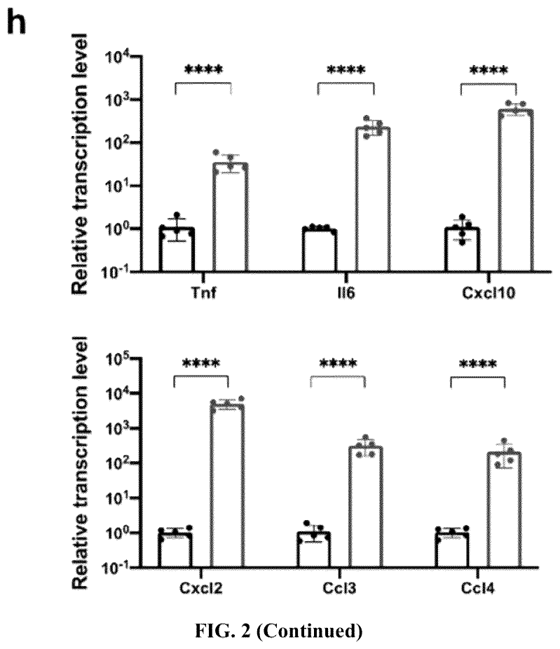

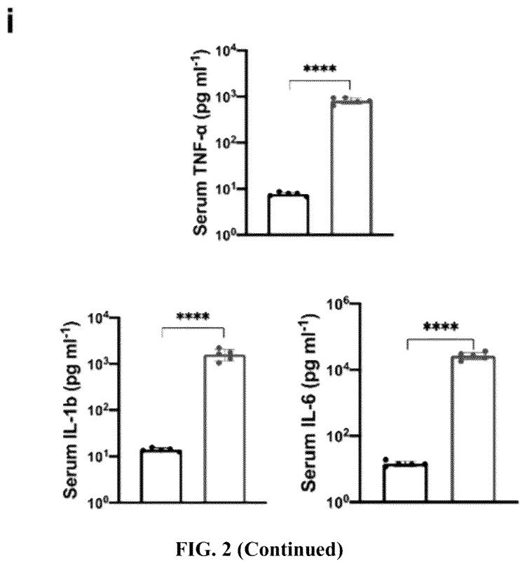

Given the capacity of ATCC 17978 to persist intracellularly and extracellularly, we hypothesized that the symptoms of infection were induced by a hyperactive immune response triggered by the continuous stimulation of the host immune system by Ab. To evaluate the systemic immune response induced by Ab, we analyzed the changes in the number of immune cells in the lungs and spleens of Ab-infected mice by flow cytometry. We observed enhanced infiltration of macrophages (CD11b⁺F4/80⁺) and neutrophils (CD11b⁺Ly6G⁺), yet a decreased in the size of population of lymphocytes, including T cells (CD19-CD3+), B cells (CD3-CD19+) and natural killer (NK) cells (CD3⁻NK1.1⁺) in the lungs upon Ab infection (FIG. 2, A to D). Rapid recruitment and activation of neutrophils and macrophages was observed at the early stage of Ab infection. Macrophages play a crucial role in phagocytosing Ab and releasing cytokines that recruit neutrophils to control infection¹². Previous studies showed that neutrophils were critical for Ab infection control, so that mortality rate and infection severity increased sharply in neutrophil-depleted mice¹³⁻¹⁵. In addition, lymphocytopenia is a predictor of systemic inflammatory responses caused by bacterial infection, and is characterized by reduction of T, B, and NK cells in the lungs of Ab-infected mice, in which the CD206 MFI levels were found to decrease and the CD86 MFI levels on the surface of CD11b⁺F4/80⁺ cells increased, indicating that these cells were polarized to a pro-inflammatory M1 phenotype (FIG. 2B).

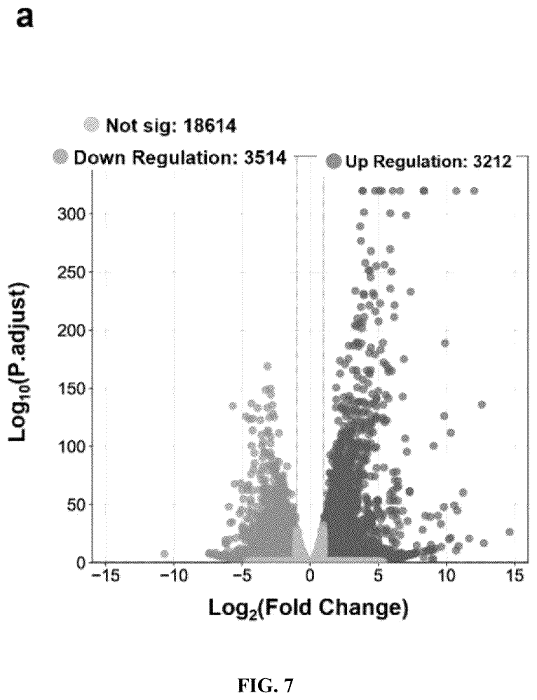

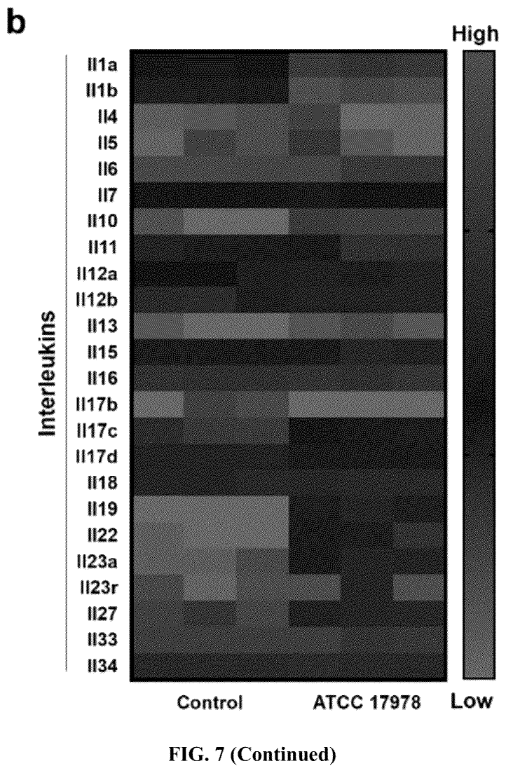

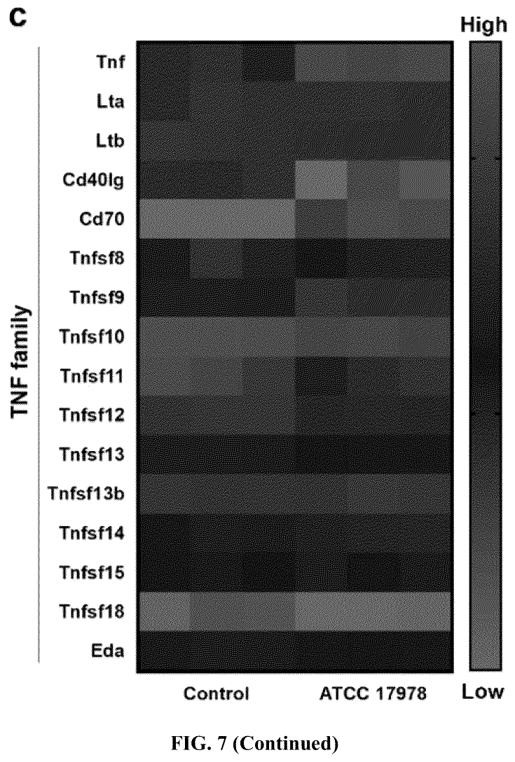

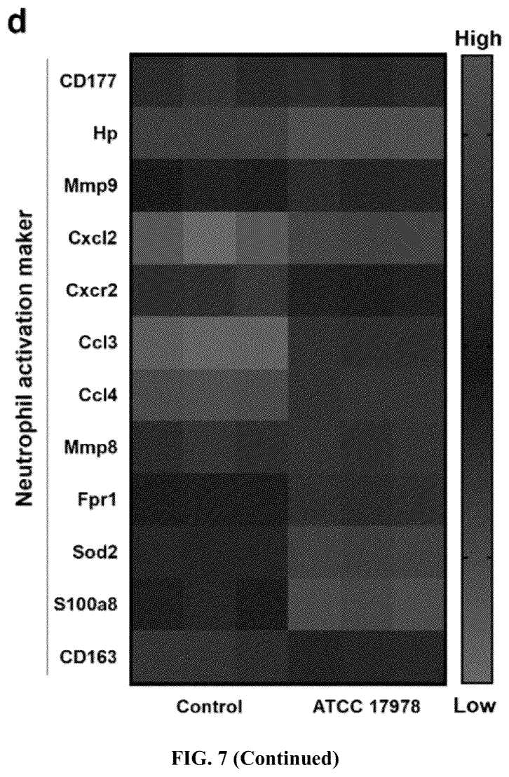

We then investigated the differences between the gene transcription levels of cells collected from healthy and infected mice using RNA-seq. It was found that a total of 3212 genes significantly up-regulated and 3514 genes were down-regulated after Ab infection (FIG. 7A). KEGG enrichment analysis showed that metabolic pathways that were highly responsive to infection were those involved in expression of cytokines and chemokines in innate immune system marked with red (FIG. 2E). Moreover, a relatively high expression level of the proinflammation (M1 polarization) markers (FIG. 2F), chemokines (FIG. 2G), interleukins, and TNF family and neutrophil activation markers (FIG. 7, B to D) was depicted by constructing a heatmap. Several differentially expressed pro-inflammatory marker genes were selected for verification of their expression level by RT-qPCR (FIG. 2H). The levels of cytokines (TNF-α, IL-1b, and IL-6) in mouse serum were found to have increased by more than 100 folds at 12 hpi (FIG. 2I). These findings are consistent with the observation of proinflammatory M1 macrophage polarization. All in all, our data suggest that Ab infection could systematically trigger significant macrophage and neutrophil recruitment, lymphopenia, as well as dysregulation of cytokine production and excessive inflammatory response.

Revelation of Functional Role of Macrophages in Mediating Excessive Inflammatory Response During Ab Infection by scRNA-Seq

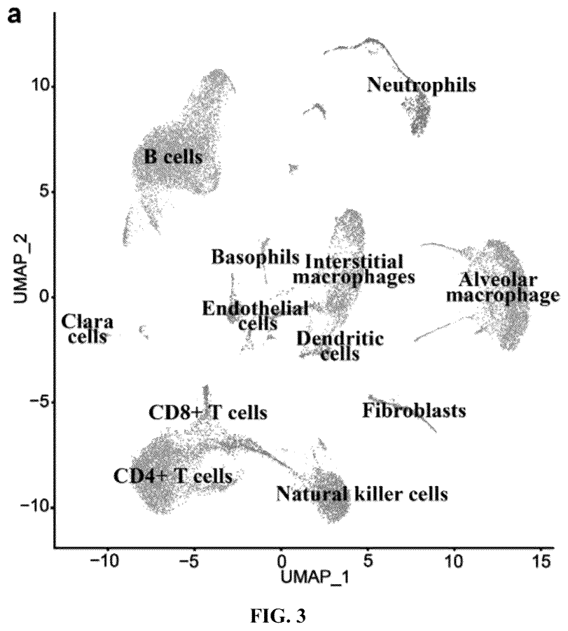

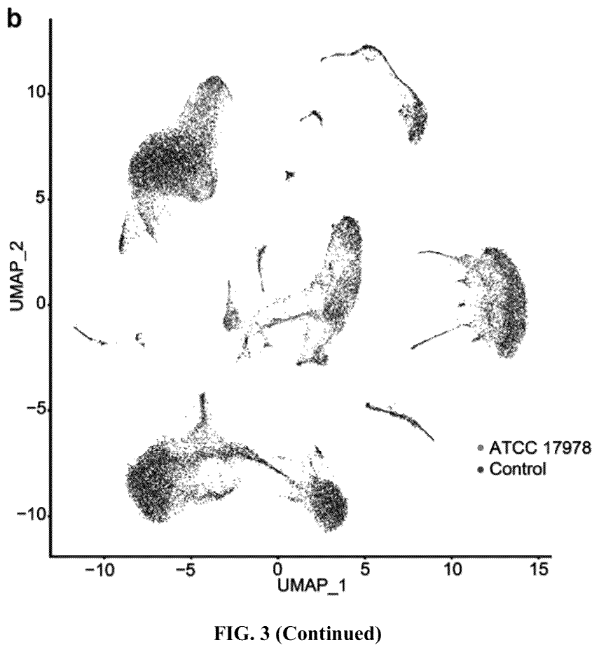

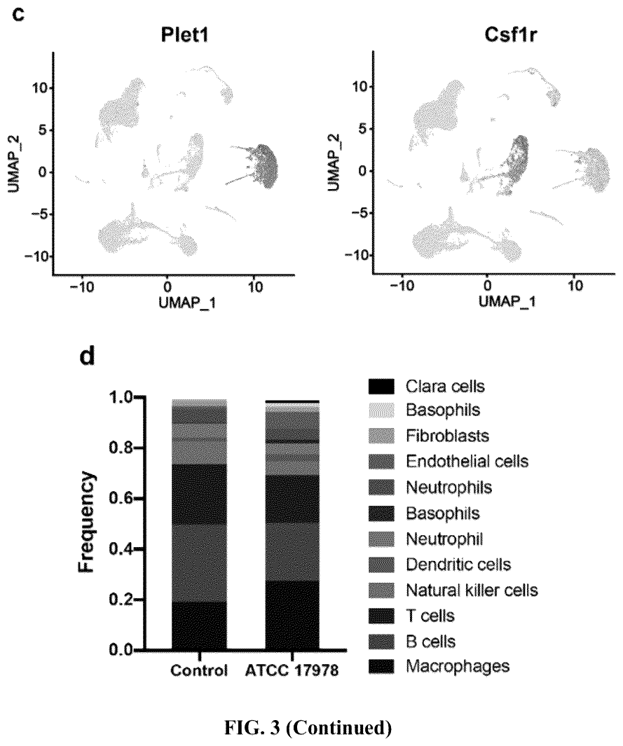

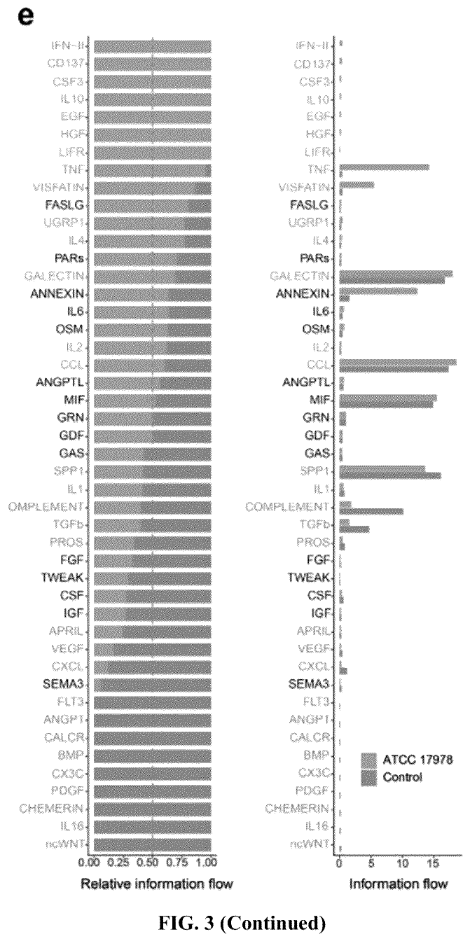

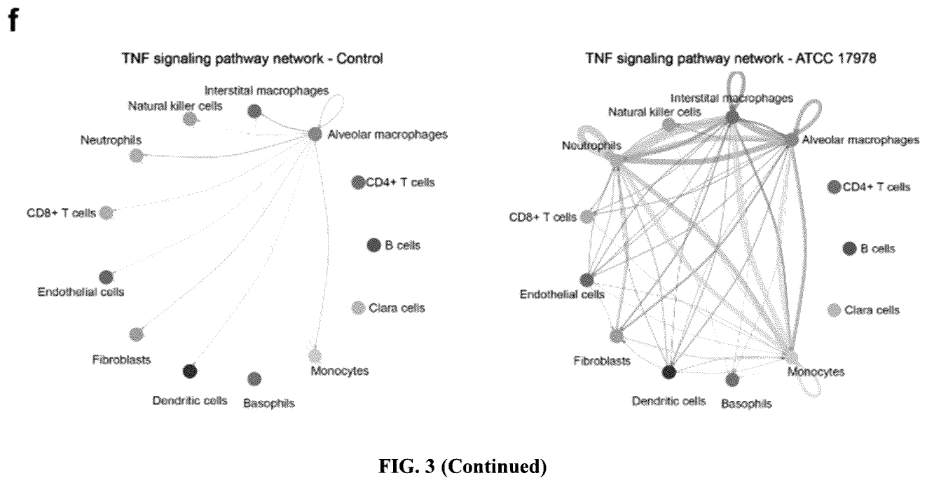

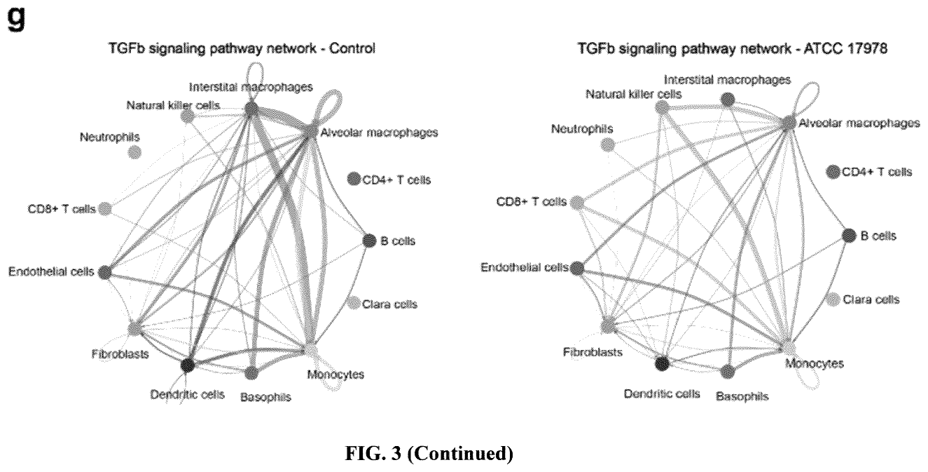

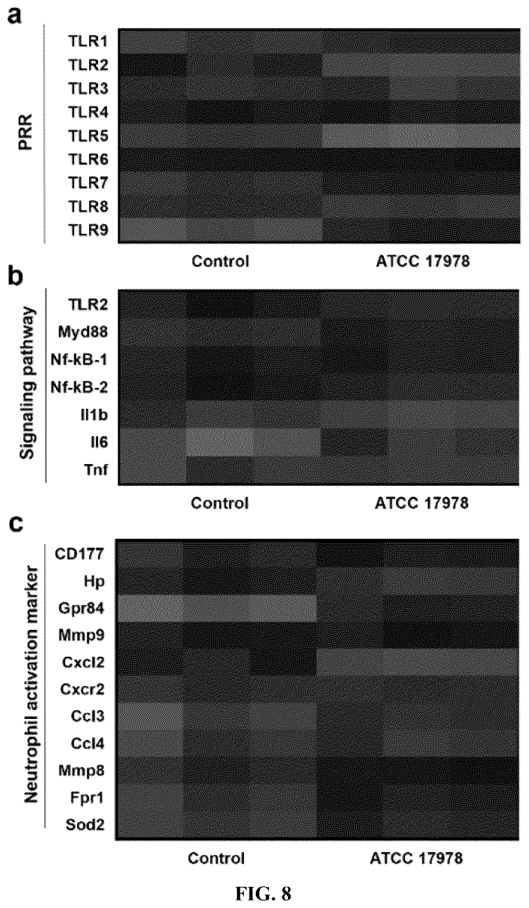

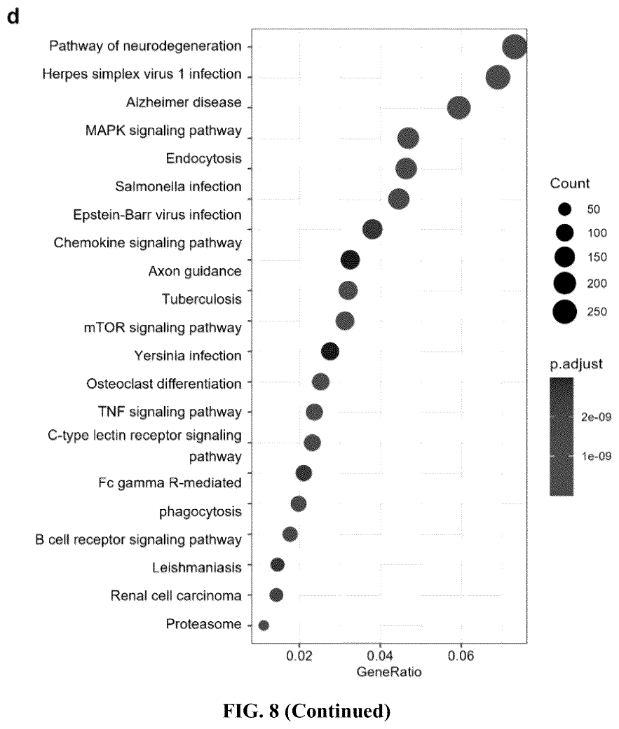

Given the changes in the population structure of immune cells and elicitation of a strong inflammatory response during Ab infection, comprehensive analysis of the Ab-induced excessive immune response was conducted by performing scRNA-seq. Through the use of the unified single-cell analysis pipeline, a cellular landscape was generated to obtain the gene expression profile of 20 774 cells, including 11 046 from healthy mice and 9 728 from ATCC 17978-infected mice. The data were presented by using the dimensionality reduction approach which involved uniform manifold approximation and projection (UMAP). Data of 14 main cell clusters are shown (FIG. 3, A to C). ATCC 17978 infection was found to result in an increase in the total number of macrophages, which was accompanied by a decrease in the frequency of detection of various lymphocyte compartments (T, B and NK cells); these findings are therefore consistent with the flow cytometry data (FIG. 3D). The smaller than expected number of neutrophils could be attributed to their susceptibility to degradation and lower RNA expression level when compared to other cells, which might result in an underestimation of the number of neutrophils in 10× genomics scRNA-seq¹⁶⁻¹⁸. To complement the scRNA-seq data, Ly6G⁺ neutrophils were subjected to differential expression analysis (FIG. 8, A to D).

To gain further insight into the nature of interactions between different cell clusters, cell-cell communication analysis was conducted using Cellchat¹⁹. Global communication atlas of lung cells collected from infected and non-infected mice uncovered an overall increase in the inferred number and strength of interactions between various immune cells, as well as between immune cells and non-immune cells, upon Ab infection. The information flows of each signaling pathway were determined to depict the global immune response profiles. Upon Ab infection, we observed substantial enrichment of various signaling pathways, with most being associated with the inflammatory response. Intriguingly, the information flow of the signaling pathways related to pro-inflammatory response was turned on (IFN-II, CD37) or enhanced (TNF, VISFATIN), while that of the anti-proinflammatory signaling pathways decreased (TGFb) (FIG. 3E). These data indicate that inflammatory microenvironment may critically contribute to progression of Ab-induced sepsis. Based on this finding, we investigated the specific cellular components crucial to the relevant pathways. In the TNF signaling pathway, the total signal intensity of cell interactions in Ab-infected individuals was found to increase when compared to healthy individuals, especially in alveolar macrophages (AMs), interstitial macrophages (IMs), and neutrophils, where significantly increased signals via paracrine and autocrine was observed (FIG. 3F). In the TGFbeta signaling pathway, interactions of two kinds of macrophages with other immune cells were strongly attenuated, and autocrine signaling was completely lost in IMs upon Ab infection, whereas interactions of natural killer cells and neutrophils with other cells were enhanced (FIG. 3G). Neutrophils, IMs, and AMs exhibited notable changes in signal intensities in the inflammatory signaling pathway when interacting with other cells; in particular the signal intensities of IMs and AMs exhibited consistent proinflammatory trends upon Ab infection. Due to the limited number of neutrophils detectable in scRNA-seq 16-18, alveolar and interstitial macrophages were regarded as key cellular components that mediate the Ab-induced hyper-inflammatory response.

### Ab Infection Inducing Cytokine Storm in Mice by Modulating Macrophage Polarization

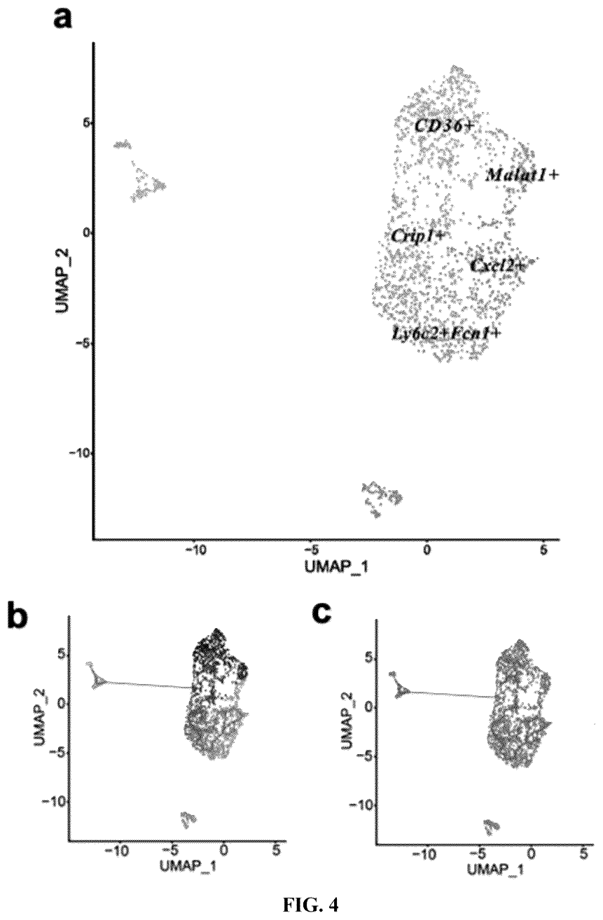

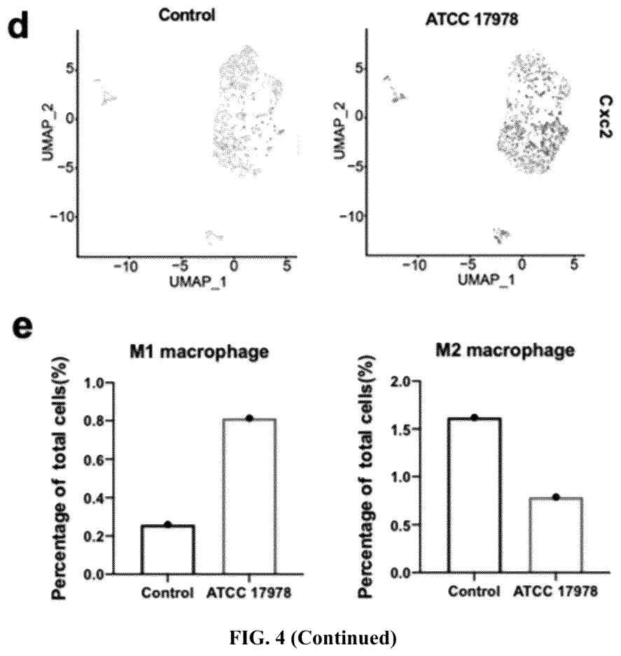

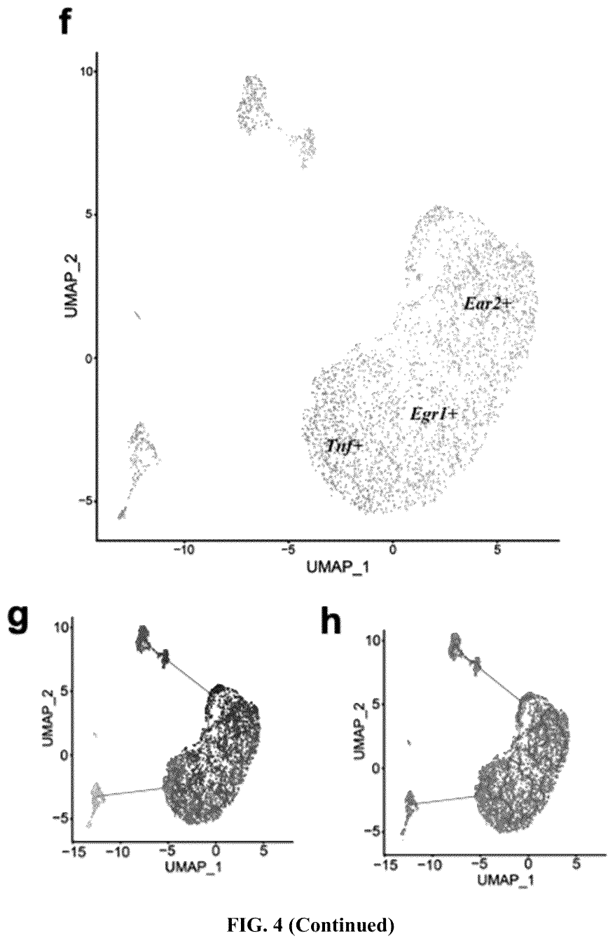

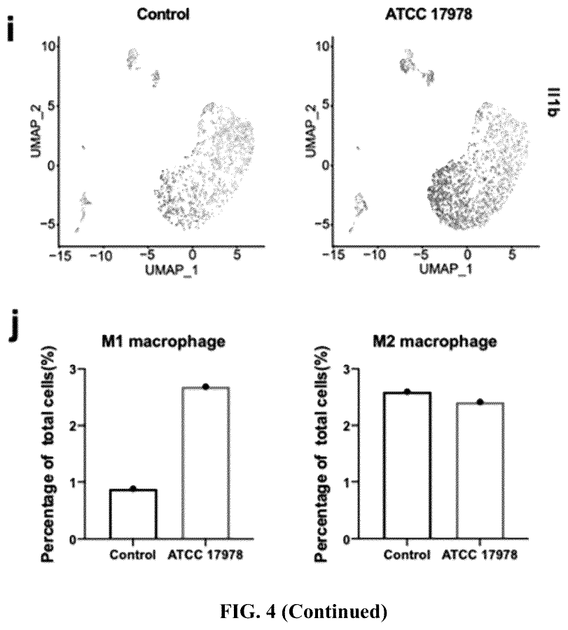

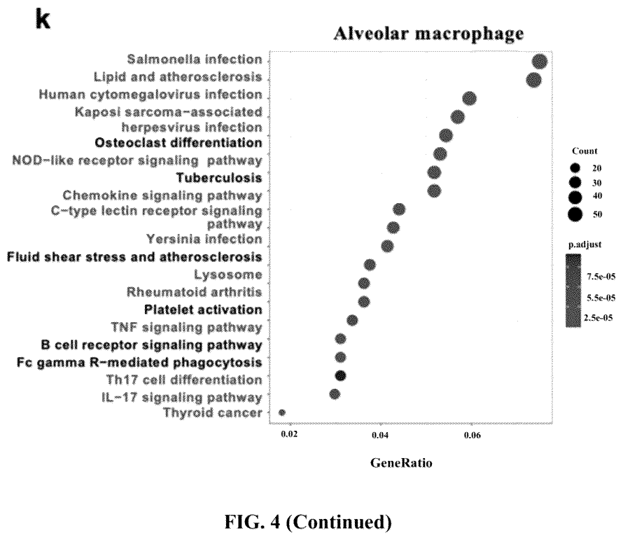

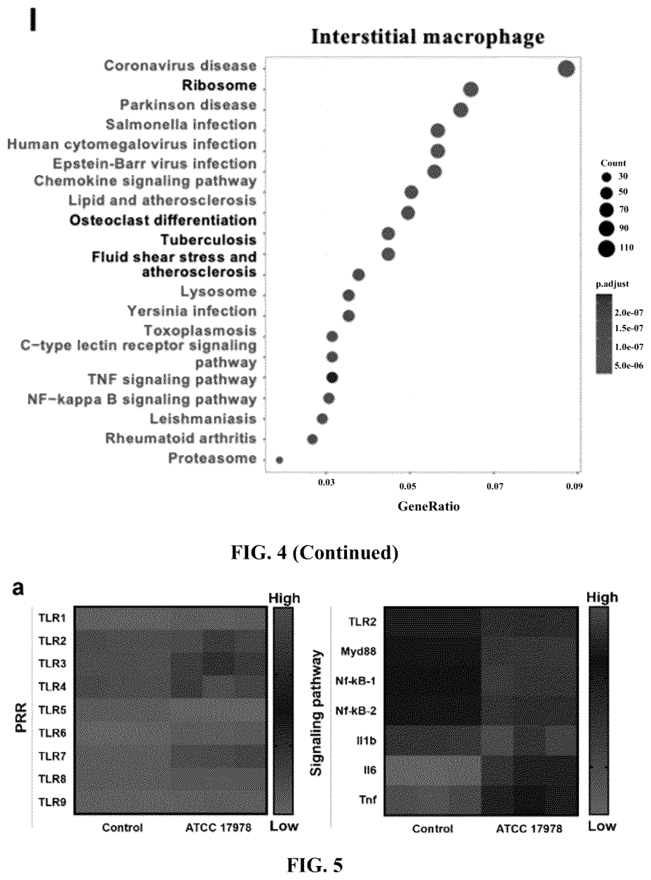

Since IMs and AMs may be the main target of Ab during the infection process, we further performed re-clustering of these two types of macrophages and subsequently constructed the cell lineages of the differentiation trajectories to determine if M1 polarization patterns along with pseudotime could be observed upon Ab infection. By setting the starting point of the trajectory as CD36⁺ M2 macrophages in AMs and Ear2⁺ M2 macrophages in IMs, gradual evolution towards Cxc12-labeled or Tnf-labeled M1 macrophages could be observed in both populations as pseudotime advanced. Such evolution process led to enhanced M1-like macrophages and decreased M2-like macrophages in AMs of Ab-infected mice when compared to healthy mice (FIGS. 4, A, B, C, and E). The number of M1 and M2 macrophages was also found increase at a similar extent among IMs subclusters after Ab infection (FIGS. 4, F, G, H and J). The findings of pseudotime trajectory analysis therefore suggest a transition from a M2 polarization state to a M1 polarization state in IMs and AMs, and that such transition became more apparent in Ab-infected animals. This conclusion is also supported by an increase in the amount of CD86 MFI and a decrease in the amount of CD206 MFI on the surface of macrophage upon Ab infection, as shown in analysis by flow cytometry (FIG. 2B).

Consistently, both AMs and IMs from Ab-infected individuals generally exhibited enhanced expression levels of M1 polarization and proinflammatory marker genes (Cxcl2, Cxcl10, Tnf, Il1b, Ptgs2, Csf3, FIGS. 4, D and I and FIGS. 8, B and C). The enrichment analysis illustrated the vast majority of the top 20 significantly activated KEGG pathways in AMs and IMs were involved in cytokine production and robust inflammatory responses (FIGS. 4, K and L). Taken together, the single-cell sequencing data further confirmed that Ab infection promotes macrophage polarization towards the M1 phenotype and resulted in formation of cytokine storm.

### Activation of TLR2/MyD88/NF-κB Signaling Pathway Promoting Ab-Induced M1 Macrophage Polarization and Eliciting Onset of Cytokine Storm

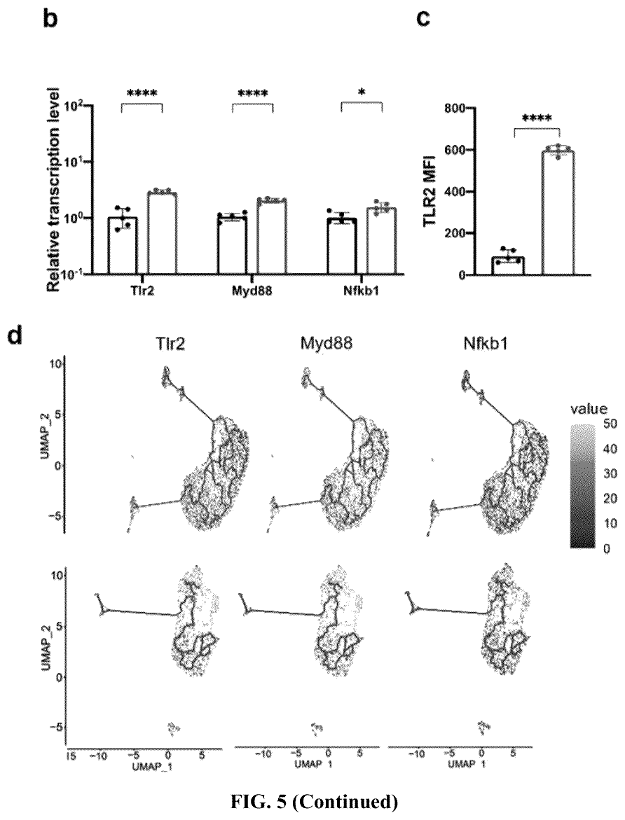

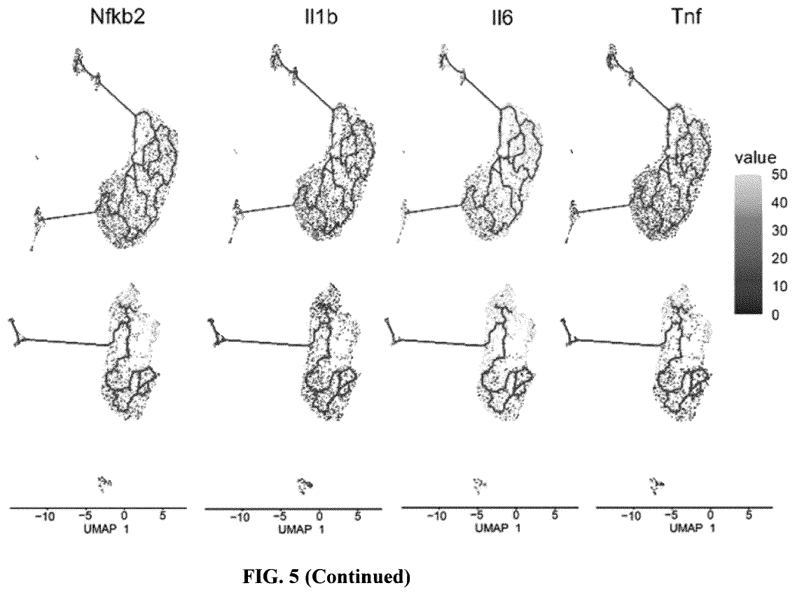

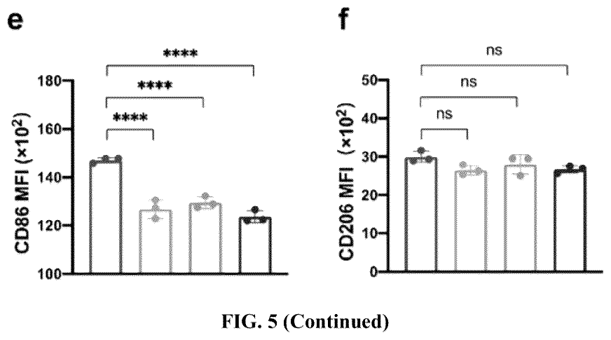

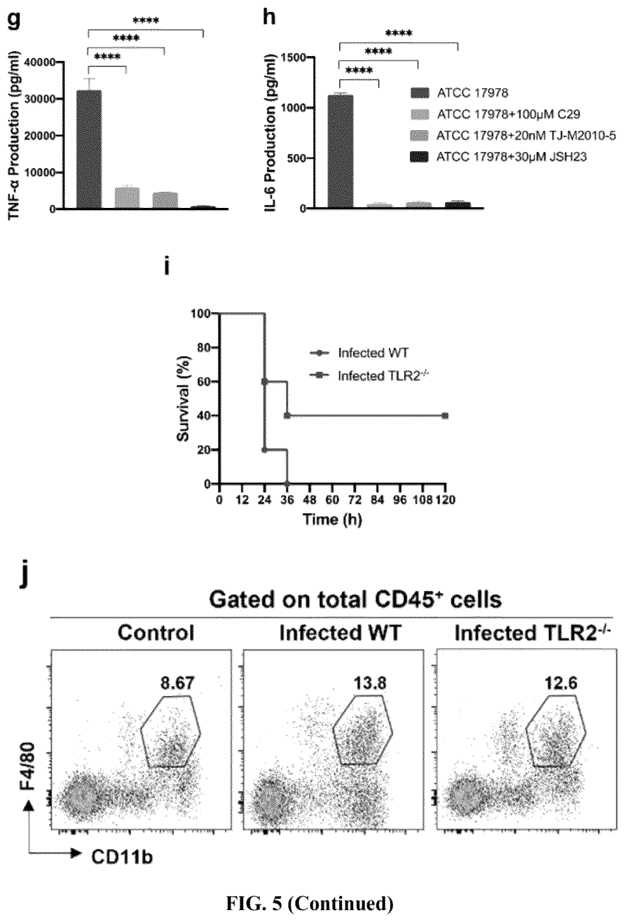

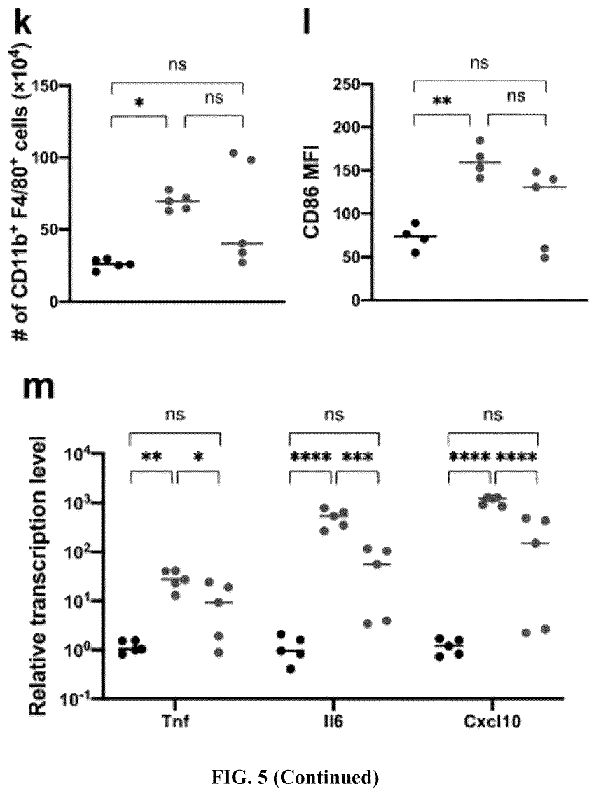

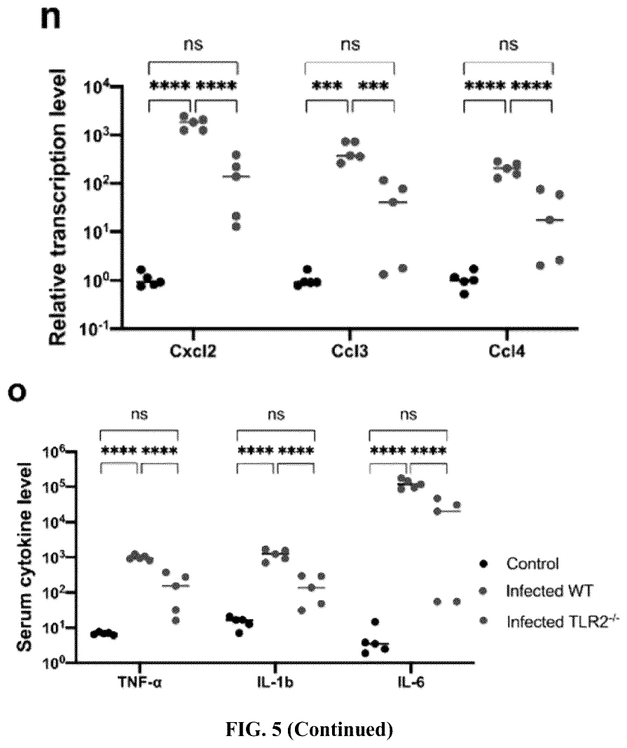

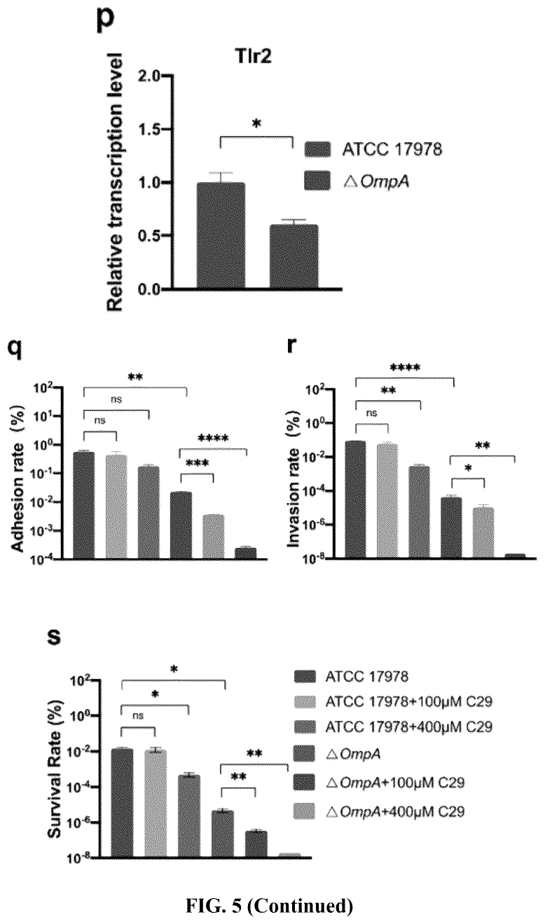

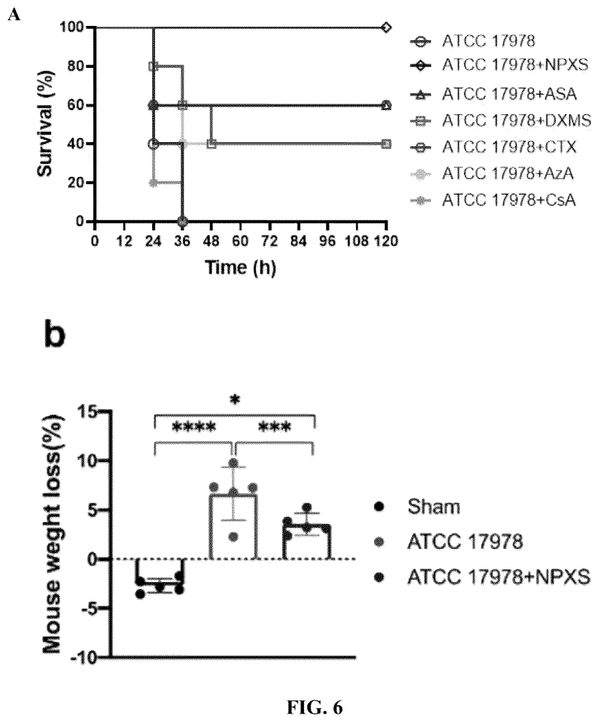

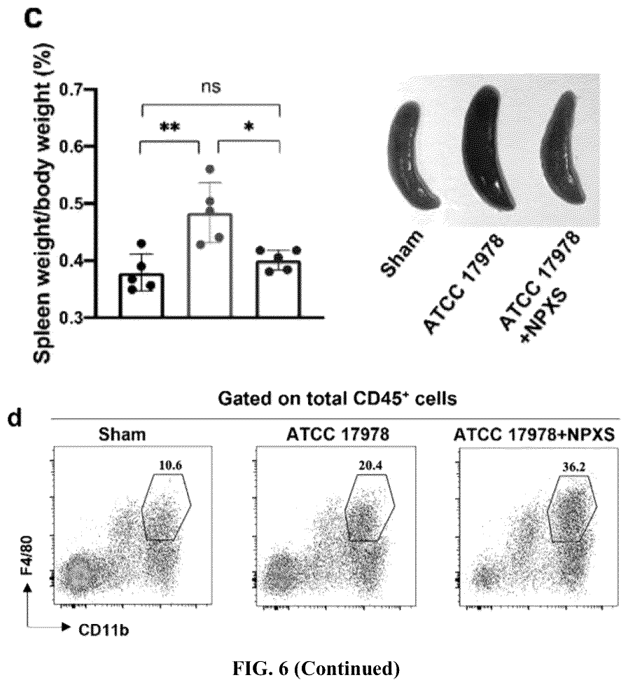

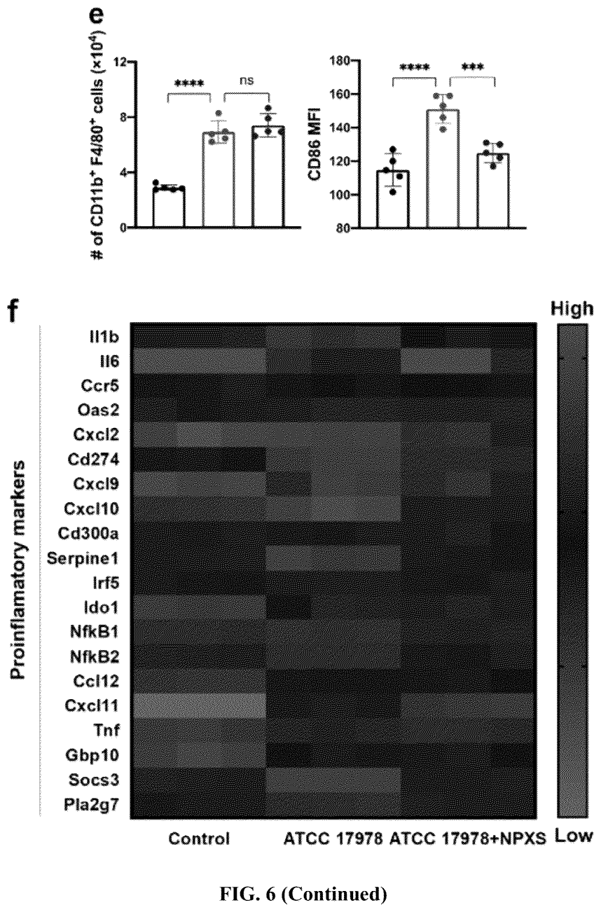

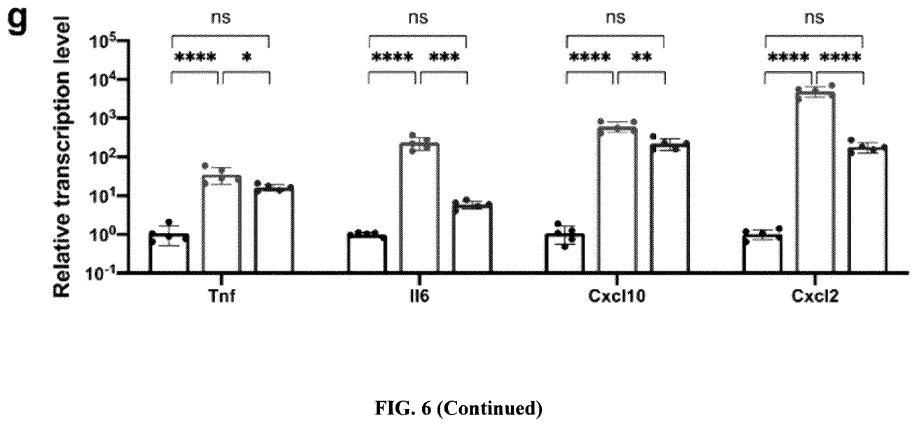

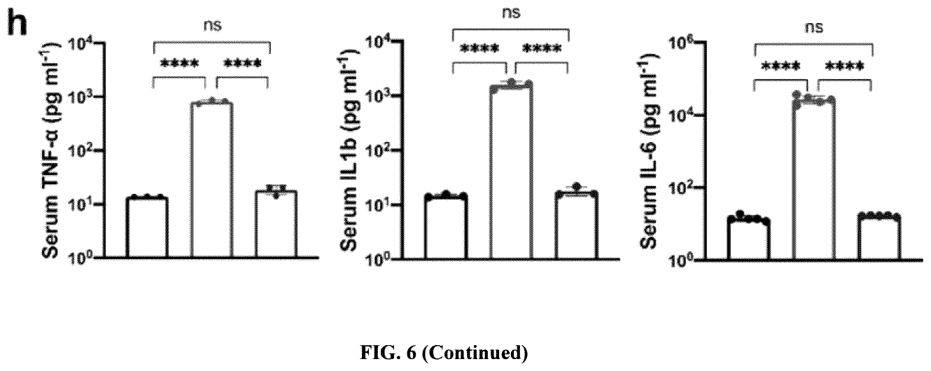

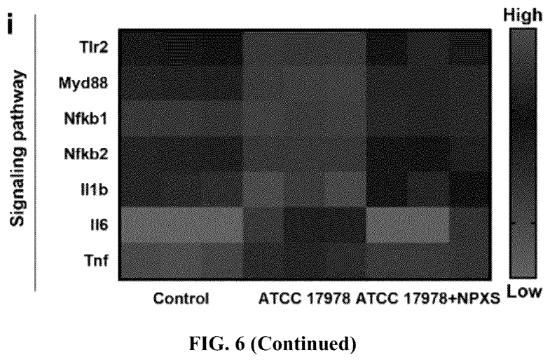

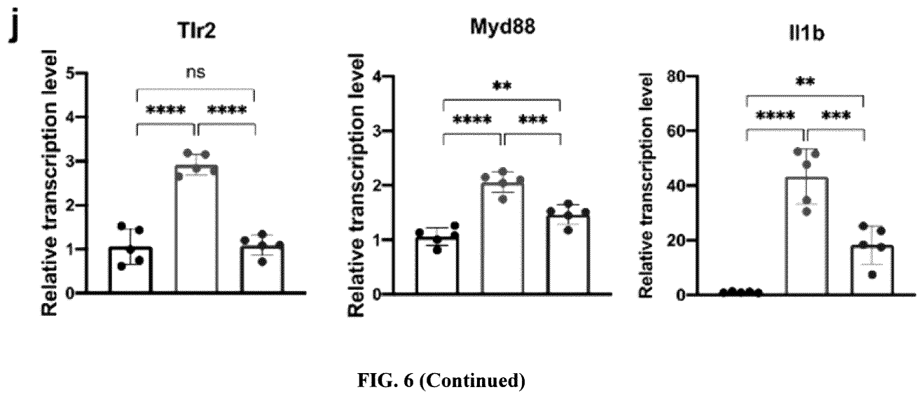

M1 polarization occurred in an inflammatory environment dominated by TLRs, which are commonly associated with activation of immune responses to combat bacterial infection²⁰. Analysis of the macrophage enhancer activity revealed that nuclear factor-κB (NF-κB) appeared to be a key transcription factor involved in M1 polarization 21. Therefore, RNA-seq and qPCR were performed to analyze the differential transcription levels of TLRs commonly shared by humans and mice; the results revealed significant up-regulation of Tlr2 expression in response to Ab infection. In addition, the transcript abundance of genes related to the TLR2/Myd88/NF-κB signaling pathway and expression of the downstream pro-inflammatory cytokines were also found to be increased in the lungs of Ab-infected mice (FIGS. 5, A and B). Tlr2 was found to be highly expressed on the surface of ATCC 17978-infected macrophages when measured by flow cytometry (FIG. 5C). Furthermore, the transcriptional levels of the aforementioned genes and the pro-inflammatory marker genes were significantly enhanced, resulting in polarization of M2 macrophages toward the M1 phenotype, suggesting that expression of these genes was also elevated in IMs and AMs during Ab infection (FIG. 5D and FIGS. 6, C and D). These findings led us to hypothesize that the TLR2/Myd88/NF-κB signaling pathway was involved in the Ab-mediated M1 polarization, which in turn caused development of cytokine storm. To test this hypothesis, RAW264.7 cells were infected with ATCC 17978 after blocking the TLR2/Myd88/NF-κB pathway with TLR2 inhibitor (C29)²², Myd88 inhibitor (TJ-M2010-5)²³ or NF-κB inhibitor (JSH-23)²⁴. We observed no difference in CD206 MFI levels, but a decrease in CD86 MFI levels when compared to cells not treated with the inhibitors (FIGS. 5, E and F), suggesting that the TLR2/Myd88/NF-κB pathway was involved in Ab-induced M1 polarization. Significant decreases were also found in IL-6 and TNF-α levels, with more than 5-fold reduction in the supernatant of RAW264.7 observed (FIGS. 5, G and H). To confirm that the cytokine storm of Ab in vivo was mediated by TLR2, TLR2 knockout (KO) and WT mice were infected with ATCC17978. The result showed a reduction of 40% in mortality in TLR2^{-/-} mice when subjected to Ab infection (FIG. 51). In addition, we observed reduced macrophage infiltration (FIGS. 5, J and K), diminished M1 polarization (FIG. 5L), decreased cytokine and chemokine expression (FIG. 5, M to O) in TLR2^{-/-} mice after Ab infection, indicating that the Ab-induced cytokine storm is TLR2-dependent. Interestingly, the living and dying populations of TLR2^{-/-} mice followed two different trajectories which correlated with the outcome of infection: infected mice that exhibit dramatically reduced cytokine production remain relatively healthy, yet a significant level of cytokine production was observed in morbid mice. Taken together, these findings further supported the theory that Ab infection could activate the TLR2/MyD88/NF-κB signaling pathway, which was a pivotal mediator of Ab-induced M1 macrophage polarization and hence also a strong trigger of cytokine production.

Previous studies have identified outer membrane protein A (OmpA) as a potential pathogen-associated molecular pattern (PAMP) recognized by TLR2 in Ab²⁵. To investigate the contribution of OmpA in onset of immune response elicited by Ab, we examined its effect on Tlr2 mRNA expression and found that the transcript abundance of Tlr2 was reduced in strain ΔOmpA (FIG. 5J). In the presence of C29, we observed reduced adhesion and invasiveness and consequently decreased survival of ATCC 17978 in macrophages. Notably, these effects were more pronounced when TLR2-inhibited macrophages were infected by the strain ΔOmpA (FIG. 5, K to M). These observations suggest that OmpA activated TLR2 on the surface of macrophages, facilitating cell adhesion and invasion by Ab.

### NPXS Rescuing Ab-Infected Mice by Attenuating Cytokine Storm

To confirm that there is a high degree of correlation between onset of cytokine storm and mouse lethality upon Ab infection, immunosuppressive drugs were intraperitoneally administered to mice to attenuate cytokine production in infected mice and determine if mortality rate could be reduced. Another purpose of this experiment is to test whether it is feasible to develop a novel approach for treatment of Ab infection by suppressing the strong inflammatory response elicited by Ab. The therapeutic drugs utilized in this study comprised naproxen (NPXS), acetylsalicylic acid (aspirin, ASA), dexamethasone (DXMS), azathioprine (AzA), and cyclosporine A (CsA). Apart from CsA treatment, which could not improve the survival of ATCC17978-infected mice, the administration of other immunosuppressants prolonged their survival. The survival rates of ATCC17978-infected mice subjected to ASA, AzA, and DXMS treatment were 60%, 40%, and 40% at 24 h, 36 h and 48 h, respectively, while NPXS protected 100% of the infected mice from death within 120 h, indicating its superior effectiveness compared to other treatments (FIG. 6A). Moreover, the extent of body weight loss was found to decrease from 6.7% to 3.6% and that of splenomegaly decreased from 0.48% to 0.38% on average upon NPXS treatment (FIGS. 6, B and C). Although NPXS treatment did not result in decrease in infiltration of CD11b⁺F4/80⁺ macrophages, it effectively attenuated CD86 MFI levels, thereby inhibiting the macrophage polarization towards the M1 phenotype (FIGS. 6, D and E). Consistently, the transcript levels of most pro-inflammatory genes in Ab-infected mice were significantly lower after NPXS treatment (FIGS. 6, F and G). NPXS was also found to reduce serum cytokine production (TNF-α, IL-6 and IL-1b) in Ab-infected mice by more than 50-fold (FIG. 6H).

Non-steroidal anti-inflammatory drugs (NSAIDs) such as NPXS and ASA exert potent anti-inflammatory and immunosuppressive effects by inhibiting the cyclooxygenase (COX) isoforms COX-1 and COX-2²⁶. Notably, the most effective drug NPXS is a nonselective inhibitor of COX-1 and COX-2, converting arachidonic acid into prostaglandins (PG), such as PGD 2, PGE 2, and so on²⁷. Both the COX-2/PGE 2 and TLR/MyD88 signaling pathways are involved in the inflammatory response, and interactions between these two different pathways are crucial for regulation of the inflammatory microenvironment²⁸. In addition, deletion of genes that encode COX-1/COX-2 altered the response to TLR activation in a TLR-specific manner²⁹. RNA-seq analysis revealed that expression of the COX1 (Log₂FC: 0.4194) and COX2 (Log₂FC: 0.2429) genes were suppressed in ATCC 17978-infected mice after NPXS treatment, which resulted in further reduction in the relative expression of TLR2/Myd88/NF-κB signaling pathway-related genes and ultimately attenuating the cytokine storm induced by Ab infection (FIGS. 6, I and J). These findings also suggested a potential role for the TLR2/Myd88/NF-κB signaling pathway in regulating the cytokine storm induced by M1 polarization. Taken together, these data revealed that Ab-induced mortality was attributed to excessive cytokine production during infection, and NPXS could serve as a novel therapeutic agent to save the lives of mice from fatal Ab infection by attenuating the cytokine storm.

Ab poses a severe threat to public health. Existing approaches for treatment of Ab infections primarily rely totally on the use of antimicrobial agents. However, the increasing prevalence of MDR infections has made antibiotic treatments increasingly challenging. In this study, we provided a novel insight into the treatment of Ab infections through adopting an immunosuppressive approach. We focused on investigating the innate immune response elicited by Ab. In a mouse sepsis model, we found Ab infection led to the polarization of AMs and IMs to M1 phenotype, triggering a cytokine storm and ultimately host death. The polarization of two types of macrophages towards the M1 phenotype is primarily mediated by the TLR2/MyD88/NF-κB signaling pathway, which then triggers cytokine over-production. This excessive inflammatory response could be attenuated by NPXS, an immunosuppressant, which was found to effectively protect mice from death upon infection by Ab.

The immune response elicited by Ab is a highly complex process that involves a range of antimicrobial activities mediated by macrophages and other immune cells of the host. At the early stage of Ab infection, there is significant recruitment of macrophages due to activation of the innate immune response. Although the activities of macrophages are bactericidal, our findings show that a considerable number of Ab remained viable at 12 hpi. Previous studies revealed that ATCC 17978 exhibits a higher level of virulence by evading macrophage-mediated killing³⁰. This organism can therefore exhibit resistance to macrophages in the extracellular environment and secrete OMVs to deliver virulence factors into host cells, inducing apoptosis³¹. Intracellularly, Ab could replicate in vacuoles and escape from macrophages³². The presence of viable Ab continuously stimulates the membrane-bound pattern recognition receptor TLR2, upregulates the TLR2/MyD88 pathway, activates the transcription factor NF-κB, and promotes M1 macrophage polarization, thereby inducing over-production of inflammatory factors (IL-1B, IL-6, and TNF-α). As a result, a severe cytokine storm is triggered, producing a systemic inflammatory response syndrome which is often associated with systemic organ failure and even death of the host.

The varied inflammatory responses induced by M1 macrophages exert different effects on the host innate immunity and in turn a diverse range of clinical outcomes. Moderate polarization of M1 macrophages may confer protective effects on the host during acute infectious diseases³³. Listeria monocytogenes have been shown to trigger an M1 program that prevents bacterial phagosome escape and promotes intracellular bacterial killing³⁴. Prolonged or excessive activation of the M1 program induces a cytokine storm, which is detrimental to the host. Uncontrolled activated M1 macrophages could produce high levels of type 1 cytokines and chemokines, which contribute to inflammation in multiple organs, with potentially fatal outcome. Our findings first illustrated that Ab infection induced this uncontrolled M1 polarization and cytokine storm, which were linked to serious complications, multi-organ failure and death of the host. Cytokine storm is also known to be triggered by various viruses, bacteria, or fungi infections. A number of Gram-negative bacteria have been reported to cause the excessive inflammatory response in the host, these include Yersinia pestis ³⁵, Francisella tularensis³⁶, Pseudomonas aeruginosa³⁷, and Klebsiella pneumoniae³⁸. Hence, inhibiting hyperpolarization of M1 macrophages or excessive expression of cytokines may represent a novel approach for the treatment of infections caused by Ab and other pathogens, regardless of their antibiotic susceptibility profiles.

We showed that immunosuppressive drugs could be used to suppress the inflammatory response induced during Ab infection. Our results showed that NSAIDs exhibited high efficacy as a therapeutic agent that acts by inhibiting the Ab-triggered cytokine storm, with naproxen being the most effective. A previous study also demonstrated the effectiveness of naproxen to control the cytokine storm in chronic and severe cases of COVID-19 diseases 39. Naproxen, a nonselective inhibitor of COX-1 and COX-2, suppresses the constitutive expression of COX-1 in most cells, as well as the induced expression of COX-2 in response to inflammatory stimuli 40. In this study, we found that the anti-inflammatory potency of aspirin recorded during Ab infection was inferior to that of naproxen. COX-2 has been regarded as the most suitable target for anti-inflammatory drugs. Also, it was shown that the therapeutic anti-inflammatory effect of NSAIDs is achieved by inhibiting COX-2, whereas the adverse side effects are primarily associated with COX-1 inhibition 41. The superior inhibitory effect of naproxen on COX-2 compared to aspirin results in evidence of disparity in the anti-inflammatory effects and potency of the two drugs. In addition, the effect of naproxen is more long-lasting and fewer doses are required for treatment.

Our study has some limitations. First, although interstitial and alveolar macrophages act as main targets in the cytokine storm induced by Ab infection, other immune populations involved in this process, such as T cells, B cells, neutrophils, and natural killer cells, still need to be further explored. Additionally, our study revealed that AbOmpA can trigger TLR2 activation on the surface of macrophages, enhancing cell adhesion and invasion. However, more supporting evidence is required to determine whether AbOmpA can strongly induce M1 polarization and the associated inflammatory responses via upregulating TLR2/MyD88/NF-κB pathway in the future research. Last, C7BL/6 mice were used in our study, which may not may not perfectly model Ab infection of humans. Certainly, the insights gleaned from animal experiments are valuable for understanding the immune response triggered by Ab in humans and pave the way for potentially introducing the naproxen into clinical use through more clinical trails. Given that anti-inflammatory drugs such as naproxen can effectively protect mice from potentially fatal Ab infection, this drug can be considered as a promising therapeutic agent for treatment of human infections in the future.

### Materials and Methods

### Bacterial Strains and Culture Condition

A. baumannii strain ATCC 17978 was purchased from the American Type Culture Collection (VA, USA), and was incubated in LB broth and agar plates at 37° C. unless stated otherwise. When required, 50 μg/mL of cloxacillin was added for selection purposes. The bacterial strains and plasmids employed in this study are shown in FIG. 10.

### Mice

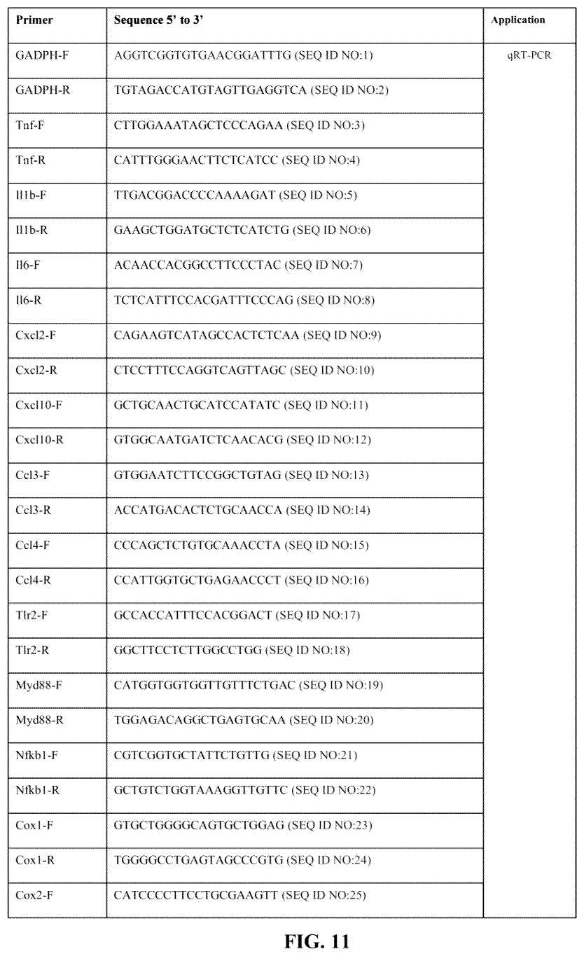

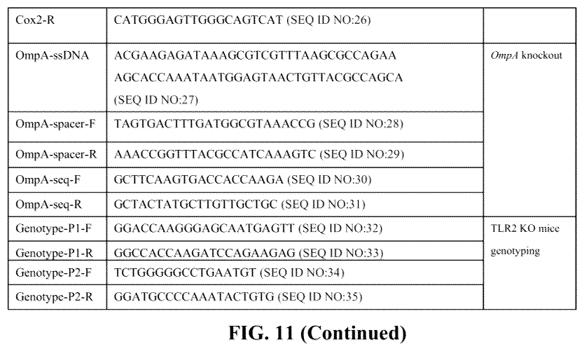

C57BL/6 and TLR2^{-/-} mice (5-7 weeks old) were employed for animal experiments. C57BL/6 mice were provided by the Laboratory Animal Research Unit of the City University. TLR2^{-/-} mice were purchased from Shanghai Model Organisms Center (Shanghai, China) and were bred in-house at the Hong Kong Polytechnic University Shenzhen Research Institute. Upon weaning, TLR2^{-/} mice of the same sex were randomized into standard-density cages and housed at 21° C. under 12:12 dark/light cycles, with access to enough water and food. The genotype of KO mice was identified by PCR with primers listed in FIG. 11. All animal experiments were approved by the City University of Hong Kong and Hong Kong Polytechnic University Shenzhen Research Institute.

### Mouse Sepsis Infection Model

The mouse infection model was employed to detect the immune response induced by Ab in vivo. Mice of the same sex were randomly divided into several groups (5 animals per group) and then infected by 7.5×10⁷ CFU of Ab test strain by intraperitoneal administration. The body weight of all mice was recorded before infection and at 12 hours post-infection. Survival of the test animals was determined by recording the mortality rate during the 120-hour experiment. At 12 hpi, the mice were sacrificed, and the organs were collected and homogenized for bacterial load measurement and analysis by flow cytometry, RNAseq, scRNAseq and qRT-PCR assay. The serum of the mice was collected for ELISA detection. Animal experiments were conducted at least twice to assess the consistency of the data.

### Collection of Spleen and Lung Single Cell

In order to collect lung cells, the entire lung sample was minced into a homogenous paste by scalpel and digested in HBSS containing 1×HEPES and 0.4 mg/ml Collagenase I for 1 hour at 37° C. The digested tissues were mashed through a 70 μm strainer to obtain lung cell suspension. Cells from spleens were pushed through a 70 μm strainer and allowed to fall into RPMI 1640 medium with 5% fetal bovine serum (FBS). ACK lysing buffer was used to lyse the erythrocyte 42.

### Determination of Bacteria Burden In Vivo

To measure the total bacterial load in different organs, including the lung, spleen, liver, kidney homogenate, and blood samples, serial dilutions were prepared and plated onto agar plates containing 50 μg/mL cloxacillin. To further assess the number of intracellular bacteria in vivo, the lungs and spleens single cell suspension was washed with PBS twice and suspended in RPMI 1640 medium containing 5% FBS with 300 μg/mL gentamicin to eliminate the extracellular bacteria by incubation at 37° C. for 2 h. The cells were then lysed with 0.2% TritonX-100 and the cell lysates were also spread onto the agar plates to count the number of phagocyted bacteria.

### Flow Cytometric Analysis

Single lung and spleen cells isolated from the test mice were incubated with different combinations of fluorochrome-conjugated antibodies against mice: CD45, CD11b, Ly6G, F4/80, CD206, CD86, CD3, CD19, CD4, CD8, NK1.1; appropriate isotype controls were acquired from Biolegend. Ghost Dye™ violet 510 (Tonbo Biosciences, CA, USA) or Propidium iodide (Sigma-Aldrich, MI, USA) was used to exclude dead cells. Flow cytometric analyses were carried out by using a FACSCelesta™ flow cytometer (BD Biosciences, CA, USA); data analysis was conducted by FlowJo software (Version 10.8.1, Treestar, CA, USA).

### RNA Extraction and Real-Time Quantitative PCR Assay

Total RNA was extracted from the homogenized lung samples or RAW264.7 cells (ATCC, CA, USA) upon Ab infection, utilizing the TRIzol™ method (Thermo Fisher Scientific, MA, USA). Turbo DNA-free™ Kit (Invitrogen, CA, USA) was employed for removal of DNA contaminants. 1 μg purified mRNA was reverse transcribed into cDNA using SuperScript™ III First-Strand Synthesis SuperMix Kit (Invitrogen), with a 1:10 dilution for subsequent qRT-PCR analysis. The qRT-PCR was then performed on an ABI QuantStudio 7 Flex Real-Time PCR System (Applied Biosystems, CA, USA) by the PowerUP SYBR Green master mix (Applied Biosystems). The primers used are shown in FIG. 11. The target gene transcription levels were quantified by the comparative Ct method and normalized to the expression level of glyceraldehyde-3-phosphate dehydrogenase (GADPH).

### RNA Sequencing

Total RNA extracted from lung samples was sequenced by Novogene Technology (Hong Kong SAR, China). Bioanalyzer 2100 system was used to measure RNA integrity (Agilent Technologies, CA, USA); the NEBNextUltra RNA Library Prep Kit (NEB, CA, USA) was employed to construct the cDNA libraries for Illumina sequencing. The index-coded samples clustering was carried out on a cBot Cluster Generation System by the TruSeq PE Cluster Kit v3-cBot-HS (Illumia, CA, Illumia). The libraries were sequenced on the Illumina Novaseq platform with paired-end 150 bp reads. HISAT2 was used for sequencing reads alignment. Fragments per gene were counted based on exon regions with FeatureCounts v.1.6.243. Differential mRNA level analysis was carried on by the DESeq2 R package. Unigenes with P.adjust<0.05 and |Log2Fold Change|>1 could be described as differentially expressed genes. Gene Enrichment Analysis was conducted for gene ontology enrichment analysis. The up/down-regulated DEGs were visualized by volcano plots.

### Serum Collection

Blood was collected from the test mice by orbital bleeding and clotted at RT for 1 h. Blood was subsequently subjected to centrifugation at 10,000 g for 25 min at 4° C. Serum was divided into tubes and stored immediately at −80° C. until cytokine analysis.

### Cytokine Analysis

The enzyme-linked immunosorbent assay (ELISA) was used to perform cytokine analysis in mouse serum and cell supernatants. Mouse serum was extracted as described above. Pre-treated macrophage cell supernatants were harvested and centrifuged to remove impurities. The levels of IL1β, IL6, and TNF-α were measured by using the IL-1 beta, IL-6 and TNF alpha Mouse Uncoated ELISA Kit (Thermo Fisher Scientific) according to the manufacturer's protocols, respectively.

### Single-Cell RNA Sequencing and Data Processing

The single cell suspension extracted from mouse lung samples was sent to BGI Genomics (Hong Kong SAR, China) for 10× genomics single-cell sequencing. Briefly, the 10× Genomics® Chromium™ System Single Cell 3′ Library Construction Kit v3 and Next GEM Single Cell 3′ Library Construction Kit v3.1 were employed to build premade libraries. After quality analysis of premade libraries, they were converted to DNBs (DNA Nanospheres) for sequencing using BGI's proprietary DNBseq™ NGS technology. De-multiplexing, barcode processing, and single-cell 5′ Unique Molecular Identifier (UMI) enumeration were performed by using the Cell Ranger Software Suite (v.3.1.0). The valid cells were filtered based on gene number, UMI count, and gene percentage of mitochondrial.

### Dimensionality Reduction and Clustering

The filtered gene barcode matrix for all samples was integrated and normalized by Seurat v.4.3 with default parameters 44. Highly variable genes were verified by the “vst” method within the Seurat FindVariableFeatures function. The variables “percent.mito” and “nCount_RNA” were regressed in the scaling step. PCA was conducted with highly variable genes. To visualize the cells, UMAP was carried out on the top—principal components. Also, the resolution was set to 0.2 to perform graph-based clustering analysis on PCA-reduced data. All clusters were subjected to differential gene expression analysis by FindAllMarkers to verify the marker genes concerned. Marker genes of each cluster were selected according to a normalized RNA expression value (RNA expression values>0.25 log-fold higher than the mean expression value and detectable expression in >25% of all cells) to annotate fine clusters. Avelar macrophages and intestinal macrophages were also re-integrated and re-clustered in the same manner.

### Cell-Cell Communication Analysis

The CellChat (v1.6.1) package 19 is utilized to infer, analyze and visualize cell-cell communication between various immune cells, using the CellChat database (github.com/sqjin/CellChat) as reference. For cellular interaction analysis, the expression levels related to the total number of reads were calculated and mapped to the same set of coding genes across all transcriptomes. Expression values were averaged in each cell sample or single-cell cluster. Differential analysis of intercellular communication between infected and non-infected groups was performed by calculating and comparing the information flow of each signaling pathway, defined as all communication probabilities between all pairs of cell populations in the inferred network.

### Pseudotime Trajectory Analysis

Trajectory analysis was conducted by Monocle version 3⁴⁵ to study the relationship between trajectories and macrophage subpopulations, after specifying the corresponding cell as the root node. We utilized the ‘plot_cells’ function to visualize the pseudotime trajectory and ordered all cells, as well as cells from healthy or Ab-infected donors, onto the trajectory.

### Differentially Gene Expression and KEGG Pathway Enrichment Analysis

The analysis of differential gene expression was conducted by utilizing the FindAllMarkers in Seurat. The one-tailed Wilcoxon rank sum test was carried out, and p-values were adjusted for multiple testing by Bonferroni correction. Differentially expressed genes (DEGs) with adjusted P value<0.01 were used for KEGG pathway enrichment analysis by the R package clusterProfiler²⁴.

### Mouse Ly6g+ Neutrophils Isolation

Lung single-cell suspensions were sorted on a MojoSort™ magnet through the use of the MojoSort™ Mouse Ly-6G Selection Kit (Biolegend, CA, USA) to select Ly6G⁺ neutrophils. Briefly, Ly6G⁺ cells were labeled by mixing the sample with the biotin antibody cocktail, followed by the addition of magnetic Streptavidin Nanobeads. These magnetically labeled cells were maintained by the magnetic separator. The purity of cells enriched from single cell populations was consistently >90%, which fulfilled the requirement by flow cytometry.

### Inhibitor Treatment in RAW264.7

RAW264.7 cells were pre-treated for 1 h with the culture medium, C29 (100 μM)²², TJ-M2010-5 (20 μM)²³ and JSH-23 (30 μM)²⁴ (MCE, NJ, USA) respectively, and then stimulated with Ab (MOI=5) for 6 h in the presence of the culture medium, C29, TJ-M2010-5 or JSH-23. Cell supernatants were collected for subsequent cytokine measurement. RAW264.7 cells were incubated with fluorescently labeled antibodies against mouse F4/80, CD206, and CD86 for flow cytometric analysis, following washes three times with PBS and resuspension in PBS containing 2.5% FBS.

### OmpA Knockout by CRISPR-Cas9-Based Genome Editing Platform

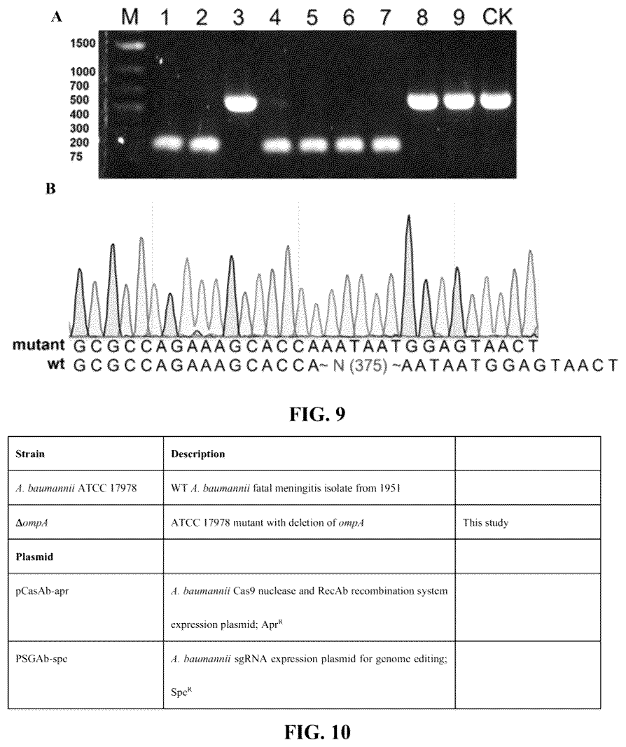

A two-plasmids genome-editing system, pSGAb-pCasAb, which combined the CRISPR-Cas9 genome cleavage system and the RecAb recombination system, was used in this study to perform efficient gene deletion in strain ATCC 17978^{46,47}. A specific 20-bp spacer sequence (sgRNA) upstream of a PAM site of OmpA was selected by the sgRNAcas9 software⁴⁸ and cloned into the pSGAb-spe plasmid (Addgene, MA, USA). Competent cells of ATCC 17978 were prepared by sub-culturing an overnight culture in LB broth, followed by culture at 37° C. with shaking until the OD₆₀₀ value reached 0.4˜0.7. The fresh exponentially growing culture was then washed with ddH₂O and then 10% glycerol which had been chilled to 4° C. The pCasAb-apr plasmid (Addgene, MA, USA) was then transformed into the competent cells of ATCC 17978 by electroporation. Upon being allowed to recover in LB broth for 1 h, the mixture was spread onto selective LB agar plates with 100 μg/mL apramycin. Transformants harboring the pCasAb-apr plasmid were selected and enriched by incubating at 37° C. until OD600 reached 0.1-0.15, followed by addition of IPTG to achieve 1 mM final concentration to induce expression of the Cas9 nuclease and the RecAb recombination system; the mixture was then further incubated for 2 h, followed by washing with ddH₂O and then 10% glycerol at 4° C. In the next step, the ssDNA (ATCC 17978 genome repair template by homologous recombination) and sgRNA-introduced pSGAb-spe plasmids were simultaneously transformed into ATCC 17978 carrying the pCasAb-apr plasmid by electroporation. For recovery and genome editing, the culture was incubated for another 1.5 hours before being spread onto LB agar plates with 100 μg/mL apramycin and 50 μg/mL spectinomycin. After incubation overnight, the colonies were collected and subjected to confirmation of whether the ompA gene had been deleted by PCR and nucleotide sequencing. The PCR validation results of ATCC17978 and ΔOmpA are shown in FIG. 9, and the primers and ssDNA used in this study are listed in FIG. 11.

### Bacterial Adherence, Invasion, and Survival in RAW264.7 Cell Cultures

Murine macrophage-like RAW264.7 cells were seeded into a 24-well culture plate at a density of 2×10⁵ cells per well. These cells were treated for 1 h with culture medium alone and those containing 100 μM C29 or 400 μM C29²² and then infected with A. baumannii strains (MOI=10). For cell adhesion assay, cells were washed 3 times with PBS 30 min after infection and then lysed in 1 ml 0.2% Triton X-100. To analyze the invasion rate, DMEM medium containing 300 μg/mL gentamicin supplemented with 10% FBS was added 1 hour after infection and incubated for an additional 2 hours to eradicate all extracellular bacteria. In the next step, the cells were washed 3 times with PBS and lysed in 1 ml 0.2% Triton X-100 to release the invading bacteria from the infected cells. For RAW264.7 intracellular survival assay, cells were incubated in culture medium with 300 μg/mL gentamicin for 2 h; after washing with PBS, culture medium containing 15 μg/mL gentamicin was added to control the growth of intracellular bacteria released from lysed macrophages. The cells were washed and lysed in 1 ml 0.2% trionX-100 after an extended 4 h of incubation. The lysate was serially diluted and spread on LB agar plates.

### Drug Treatment In Vivo

To test the effect of NSAIDs treatment, the mice were administered intraperitoneal injections of 50 mg/kg NPXS and 100 mg/kg ASA 1 h before ATCC 17978 infection; a group of mice that served as control received equivalent volumes of PBS. The survival rate, health status, and weight of the test animals were recorded during the next 120 hours.

### Statistical Analysis

All experiments were conducted at least twice, with each experiment involving a minimum of three biological replicates. The statistical data were plotted by using GraphPad Prism 8; differential analysis was carried out by performing Tukey's correction for multiple comparisons. The Log-rank (Mantel-Cox) test was used for comparing survival rates in animal experiments.

### REFERENCE

ADDIN EN.REFLIST 1 Giammanco, A., Calà, C., Fasciana, T. & Dowzicky, M. J. Global assessment of the activity of tigecycline against multidrug-resistant Gram-negative pathogens between 2004 and 2014 as part of the tigecycline evaluation and surveillance trial. Msphere 2, e00310-00316 (2017).
2 Rolain, J.-M. et al. Real-time sequencing to decipher the molecular mechanism of resistance of a clinical pan-drug-resistant Acinetobacter baumannii isolate from Marseille, France. Antimicrobial agents and chemotherapy 57, 592-596 (2013).
3 Ayoub Moubareck, C. & Hammoudi Halat, D. Insights into Acinetobacter baumannii: a review of microbiological, virulence, and resistance traits in a threatening nosocomial pathogen. Antibiotics 9, 119 (2020).
4 Morris, F. C., Dexter, C., Kostoulias, X., Uddin, M. I. & Peleg, A. Y. The mechanisms of disease caused by Acinetobacter baumannii. Frontiers in microbiology 10, 1601 (2019).
5 García-Patiño, M. G., García-Contreras, R. & Licona-Limón, P. The immune response against Acinetobacter baumannii, an emerging pathogen in nosocomial infections. Frontiers in immunology 8, 441 (2017).
6 Wong, D. et al. Clinical and pathophysiological overview of Acinetobacter infections: a century of challenges. Clinical microbiology reviews 30, 409-447 (2017).
7 Noto, M. J., Becker, K. W., Boyd, K. L., Schmidt, A. M. & Skaar, E. P. RAGE-mediated suppression of interleukin-10 results in enhanced mortality in a murine model of Acinetobacter baumannii sepsis. Infection and immunity 85, 10.1128/iai. 00954-00916 (2017).
8 Kang, M.-J. et al. IL-10 protects mice from the lung infection of Acinetobacter baumannii and contributes to bacterial clearance by regulating STAT3-mediated MARCO expression in macrophages. Frontiers in Immunology 11, 270 (2020).
9 Wiersinga, W. J., Leopold, S. J., Cranendonk, D. R. & van Der Poll, T. Host innate immune responses to sepsis. Virulence 5, 36-44 (2014).
10 Sameer, A. S. & Nissar, S. Toll-like receptors (TLRs): structure, functions, signaling, and role of their polymorphisms in colorectal cancer susceptibility. BioMed Research International 2021 (2021).
11 Chen, W. Host innate immune responses to Acinetobacter baumannii infection. Frontiers in Cellular and Infection Microbiology 10, 486 (2020).
12 Liu, Z. & Xu, W. Neutrophil and Macrophage Response in Acinetobacter Baumannii Infection and Their Relationship to Lung Injury. Frontiers in Cellular and Infection Microbiology 12, 890511 (2022).
13 Van Faassen, H. et al. Neutrophils play an important role in host resistance to respiratory infection with Acinetobacter baumannii in mice. Infection and immunity 75, 5597-5608 (2007).
14 Qiu, H. et al. Role of macrophages in early host resistance to respiratory Acinetobacter baumannii infection. PloS one 7, e40019 (2012).
15 Bhuiyan, M. S. et al. Acinetobacter baumannii phenylacetic acid metabolism influences infection outcome through a direct effect on neutrophil chemotaxis. Proceedings of the National Academy of Sciences 113, 9599-9604 (2016).
16 Ratnasiri, K., Wilk, A. J., Lee, M. J., Khatri, P. & Blish, C. A. in Seminars in Immunopathology. 71-89 (Springer).
17 Ekpenyong, A. E., Toepfner, N., Chilvers, E. R. & Guck, J. Mechanotransduction in neutrophil activation and deactivation. Biochimica et Biophysica Acta (BBA)-Molecular Cell Research 1853, 3105-3116 (2015).
18 Yap, B. & Kamm, R. D. Mechanical deformation of neutrophils into narrow channels induces pseudopod projection and changes in biomechanical properties. Journal of applied physiology 98, 1930-1939 (2005).
19 Jin, S. et al. Inference and analysis of cell-cell communication using CellChat. Nature communications 12, 1088 (2021).
20 Murray, P. J. Macrophage polarization. Annual review of physiology 79, 541-566 (2017).
21 Tugal, D., Liao, X. & Jain, M. K. Transcriptional control of macrophage polarization. Arteriosclerosis, thrombosis, and vascular biology 33, 1135-1144 (2013).
22 Mistry, P. et al. Inhibition of TLR2 signaling by small molecule inhibitors targeting a pocket within the TLR2 TIR domain. Proceedings of the National Academy of Sciences 112, 5455-5460 (2015).
23 Xie, L. et al. Targeting of MyD88 homodimerization by novel synthetic inhibitor TJ-M2010-5 in preventing colitis-associated colorectal cancer. JNCI: Journal of the National Cancer Institute 108 (2016).
24 Shin, H.-M. et al. Inhibitory action of novel aromatic diamine compound on lipopolysaccharide-induced nuclear translocation of NF-κB without affecting IκB degradation. FEBS letters 571, 50-54 (2004).
25 Schweppe, D. K. et al. Host-microbe protein interactions during bacterial infection. Chemistry & biology 22, 1521-1530 (2015).
26 Wongrakpanich, S., Wongrakpanich, A., Melhado, K. & Rangaswami, J. A comprehensive review of non-steroidal anti-inflammatory drug use in the elderly. Aging and disease 9, 143 (2018).
27 Funk, C. D. Prostaglandins and leukotrienes: advances in eicosanoid biology. science 294, 1871-1875 (2001).
28 Echizen, K., Hirose, O., Maeda, Y. & Oshima, M. Inflammation in gastric cancer: Interplay of the COX-2/prostaglandin E2 and Toll-like receptor/MyD88 pathways. Cancer science 107, 391-397 (2016).
29 Kirkby, N. S. et al. Differential COX-2 induction by viral and bacterial PAMPs: Consequences for cytokine and interferon responses and implications for anti-viral COX-2 directed therapies. Biochemical and biophysical research communications 438, 249-256 (2013).
30 Lin, L. et al. Inhibition of LpxC protects mice from resistant Acinetobacter baumannii by modulating inflammation and enhancing phagocytosis. MBio 3, e00312-00312 (2012).
31 Jin, J. S. et al. Acinetobacter baumannii secretes cytotoxic outer membrane protein A via outer membrane vesicles. PloS one 6, e17027 (2011).
32 Sycz, G. et al. Modern Acinetobacter baumannii clinical isolates replicate inside spacious vacuoles and egress from macrophages. PLoS pathogens 17, e1009802 (2021).
33 Shaughnessy, L. M. & Swanson, J. A. The role of the activated macrophage in clearing Listeria monocytogenes infection. Frontiers in bioscience: a journal and virtual library 12, 2683 (2007).
34 Pfeffer, K. et al. Mice deficient for the 55 kd tumor necrosis factor receptor are resistant to endotoxic shock, yet succumb to L. monocytogenes infection. Cell 73, 457-467 (1993).
35 Fajgenbaum, D. C. & June, C. H. Cytokine storm. New England Journal of Medicine 383, 2255-2273 (2020).
36 D'Elia, R. V., Harrison, K., Oyston, P. C., Lukaszewski, R. A. & Clark, G. C. Targeting the “cytokine storm” for therapeutic benefit. Clinical and Vaccine Immunology 20, 319-327 (2013).
37 Tisoncik, J. R. et al. Into the eye of the cytokine storm. Microbiology and molecular biology reviews 76, 16-32 (2012).
38 Xu, Q. et al. Molecular mechanisms underlying the high mortality of hypervirulent Klebsiella pneumoniae and its effective therapy development. Signal Transduction and Targeted Therapy 8, 221 (2023).
39 Dayer, M. R. Analgesics Candidates for JAK-STAT Pathway Inhibition as a Probable Treat for COVID-19, Bioinformatics Study. Biomacromolecular Journal 7, 10-17 (2021).
40 Valentovic, M. (Elsevier, 2007).
41 Zarghi, A. & Arfaei, S. Selective COX-2 inhibitors: a review of their structure-activity relationships. Iranian journal of pharmaceutical research: IJPR 10, 655 (2011).
42 Yang, G. et al. Pik3c3 deficiency in myeloid cells imparts partial resistance to experimental autoimmune encephalomyelitis associated with reduced IL-1B production. Cellular & Molecular Immunology 18, 2024-2039 (2021).
43 Liao, Y., Smyth, G. K. & Shi, W. featureCounts: an efficient general purpose program for assigning sequence reads to genomic features. Bioinformatics 30, 923-930 (2014).
44 Stuart, T. et al. Comprehensive integration of single-cell data. Cell 177, 1888-1902. e1821 (2019).
45 Cao, J. et al. The single-cell transcriptional landscape of mammalian organogenesis. Nature 566, 496-502 (2019).
46 Wang, Y. et al. A Highly Efficient CRISPR-Cas9-Based Genome Engineering Platform in Acinetobacter baumannii to Understand the H2O2-Sensing Mechanism of OxyR. Cell Chem Biol 26, 1732-+ (2019). doi.org: 10.1016/j.chembiol.2019.09.003
47 Wang, Y., Wang, Z. & Ji, Q. CRISPR-Cas9-Based Genome Editing and Cytidine Base Editing in Acinetobacter baumannii. STAR Protoc 1, 100025 (2020). doi.org: 10.1016/j.xpro.2020.100025
48 Xie, S., Shen, B., Zhang, C., Huang, X. & Zhang, Y. sgRNAcas9: a software package for designing CRISPR sgRNA and evaluating potential off-target cleavage sites. PLOS One 9, e100448 (2014). doi.org: 10.1371/journal.pone.0100448

What is claimed is:

1. A method of treating a cytokine storm in a subject in need thereof, the method comprising: administering a therapeutically effective amount of a therapeutic selected from the group consisting of (+)-(S)-2-(6-methoxynaphthalen-2-yl) propanoic acid (naproxen), acetylsalicylic acid (ASA), dexamethasone (DXMS), azathioprine (AzA) and pharmaceutically acceptable salts thereof to the subject, wherein the cytokine storm is associated with an Acinetobacter baumannii infection in the subject.

2. The method of claim 1, wherein the therapeutic is naproxen or a pharmaceutically acceptable salt thereof.

3. The method of claim 1, wherein the Acinetobacter baumannii infection activates a TLR2/MyD88/NF-κB signaling pathway in the subject.

4. The method of claim 1, wherein the subject overexpresses one or more pro-inflammatory factors selected from the group consisting of IL-1B, IL-6, IL-10, IL-12, IL17a, IL23, IL-27, IFN-γ, and TNF-α.

5. The method claim 1, wherein the Acinetobacter baumannii infection activates a TLR2/MyD88/NF-κB signaling pathway in the subject; the therapeutic is naproxen or a pharmaceutically acceptable salt thereof; and administration of naproxen results in a reduction in the relative expression of TLR2/MyD88/NF-κB signaling pathway-related genes.

6. The method of claim 5, wherein the TLR2/MyD88/NF-κB signaling pathway-related genes are selected from the group consisting of toll-like receptor 2 (TLR2), myeloid differentiation primary response 88 (Myd88), nuclear factor kappa B subunit 1 (Nfkb1), nuclear factor kappa B subunit 2 (Nfkb2), Interleukin 1 beta (Il1b), interleukin 6 (Il6), and tumor necrosis factor (Tnf).

7. The method of claim 1, wherein the Acinetobacter baumannii infection is the result of antibiotic resistant Acinetobacter baumannii.

8. The method of claim 7, wherein the antibiotic resistant Acinetobacter baumannii is resistant to one or more antibacterials selected from the group consisting of aminoglycosides, fluoroquinolones, and carbapenems.

9. The method of claim 7, wherein the therapeutic is naproxen or a pharmaceutically acceptable salt thereof.

10. The method of claim 1, wherein the Acinetobacter baumannii infection results from an Acinetobacter baumannii strain selected from the group consisting of ATCC 17978, ATCC 19606, AB5075, ATCC 9955, ATCC 17904, R 477, and R 0211019.

11. The method of claim 10, wherein the therapeutic is naproxen or a pharmaceutically acceptable salt thereof.

12. The method of claim 1, wherein the Acinetobacter baumannii infection is present in one or more of a wound, a surgical site, a catheter site, blood, urinary tract, skin, lungs, or respiratory tract.

13. The method of claim 1 further comprising diagnosing the subject with an Acinetobacter baumannii infection prior to administering the therapeutically effective amount of naproxen.

14. The method of claim 1 further comprising co-administering a therapeutically effective amount of an antibacterial agent to the subject.

15. The method of claim 14, wherein the antibacterial agent is selected from the group consisting of meropenem, polymyxin E, polymyxin B, sulbactam, piperacillin/tazobactam, minocycline, tigecycline and aminoglycosides.


SEQUENCE LISTING (35 sequences)

SEQ ID NO: 1 - DNA, 21 bp, Mus musculus
  source 1..21: mol_type = genomic DNA
aggtcggtgtgaacggatttg

SEQ ID NO: 2 - DNA, 23 bp, Mus musculus
  source 1..23: mol_type = genomic DNA
tgtagaccatgtagttgaggtca

SEQ ID NO: 3 - DNA, 20 bp, Mus musculus
  source 1..20: mol_type = genomic DNA
cttggaaatagctcccagaa

SEQ ID NO: 4 - DNA, 20 bp, Mus musculus
  source 1..20: mol_type = genomic DNA
catttgggaacttctcatcc

SEQ ID NO: 5 - DNA, 19 bp, Mus musculus
  source 1..19: mol_type = genomic DNA
ttgacggaccccaaaagat

SEQ ID NO: 6 - DNA, 21 bp, Mus musculus
  source 1..21: mol_type = genomic DNA
gaagctggatgctctcatctg

SEQ ID NO: 7 - DNA, 20 bp, Mus musculus
  source 1..20: mol_type = genomic DNA
acaaccacggccttccctac

SEQ ID NO: 8 - DNA, 22 bp, Mus musculus
  source 1..22: mol_type = genomic DNA
tctcatttccacgatttcccag

SEQ ID NO: 9 - DNA, 22 bp, Mus musculus
  source 1..22: mol_type = genomic DNA
cagaagtcatagccactctcaa

SEQ ID NO: 10 - DNA, 21 bp, Mus musculus
  source 1..21: mol_type = genomic DNA
ctcctttccaggtcagttagc

SEQ ID NO: 11 - DNA, 20 bp, Mus musculus
  source 1..20: mol_type = genomic DNA
gctgcaactgcatccatatc

SEQ ID NO: 12 - DNA, 20 bp, Mus musculus
  source 1..20: mol_type = genomic DNA
gtggcaatgatctcaacacg

SEQ ID NO: 13 - DNA, 20 bp, Mus musculus
  source 1..20: mol_type = genomic DNA
gtggaatcttccggctgtag

SEQ ID NO: 14 - DNA, 20 bp, Mus musculus
  source 1..20: mol_type = genomic DNA
accatgacactctgcaacca

SEQ ID NO: 15 - DNA, 20 bp, Mus musculus
  source 1..20: mol_type = genomic DNA
cccagctctgtgcaaaccta

SEQ ID NO: 16 - DNA, 20 bp, Mus musculus
  source 1..20: mol_type = genomic DNA
ccattggtgctgagaaccct

SEQ ID NO: 17 - DNA, 19 bp, Mus musculus
  source 1..19: mol_type = genomic DNA
gccaccatttccacggact

SEQ ID NO: 18 - DNA, 18 bp, Mus musculus
  source 1..18: mol_type = genomic DNA
ggcttcctcttggcctgg

SEQ ID NO: 19 - DNA, 22 bp, Mus musculus
  source 1..22: mol_type = genomic DNA
catggtggtggttgtttctgac

SEQ ID NO: 20 - DNA, 20 bp, Mus musculus
  source 1..20: mol_type = genomic DNA
tggagacaggctgagtgcaa

SEQ ID NO: 21 - DNA, 19 bp, Mus musculus
  source 1..19: mol_type = genomic DNA
cgtcggtgctattctgttg

SEQ ID NO: 22 - DNA, 21 bp, Mus musculus
  source 1..21: mol_type = genomic DNA
gctgtctggtaaaggttgttc

SEQ ID NO: 23 - DNA, 20 bp, Mus musculus
  source 1..20: mol_type = genomic DNA
gtgctggggcagtgctggag

SEQ ID NO: 24 - DNA, 20 bp, Mus musculus
  source 1..20: mol_type = genomic DNA
tggggcctgagtagcccgtg

SEQ ID NO: 25 - DNA, 20 bp, Mus musculus
  source 1..20: mol_type = genomic DNA
catccccttcctgcgaagtt

SEQ ID NO: 26 - DNA, 20 bp, Mus musculus
  source 1..20: mol_type = genomic DNA
catgggagttgggcagtcat

SEQ ID NO: 27 - DNA, 67 bp, Mus musculus
  source 1..67: mol_type = genomic DNA
acgaagagataaagcgtcgtttaagcgccagaaagcaccaaataatggagtaactgttacgccagca

SEQ ID NO: 28 - DNA, 24 bp, Mus musculus
  source 1..24: mol_type = genomic DNA
tagtgactttgatggcgtaaaccg

SEQ ID NO: 29 - DNA, 24 bp, Mus musculus
  source 1..24: mol_type = genomic DNA
aaaccggtttacgccatcaaagtc

SEQ ID NO: 30 - DNA, 20 bp, Mus musculus
  source 1..20: mol_type = genomic DNA
gcttcaagtgaccaccaaga

SEQ ID NO: 31 - DNA, 20 bp, Mus musculus
  source 1..20: mol_type = genomic DNA
gctactatgcttgttgctgc

SEQ ID NO: 32 - DNA, 21 bp, Mus musculus
  source 1..21: mol_type = genomic DNA
ggaccaagggagcaatgagtt

SEQ ID NO: 33 - DNA, 21 bp, Mus musculus
  source 1..21: mol_type = genomic DNA
ggccaccaagatccagaagag

SEQ ID NO: 34 - DNA, 17 bp, Mus musculus
  source 1..17: mol_type = genomic DNA
tctgggggcctgaatgt

SEQ ID NO: 35 - DNA, 19 bp, Mus musculus
  source 1..19: mol_type = genomic DNA
ggatgccccaaatactgtg